(19) United States
(12) Patent Application Publication
(10) Pub. No.: US 2003/0171721 A1
(43) Pub. Date: Sep. 11, 2003

(54) Medical device

(76) Inventors: Nobuo Enomoto, Uenohara-machi (JP); Norio Miyagi, Machida-shi (JP); Koki Okanda, Uenohara-machi (JP); Mitsuji Hosoda, Uenohara-machi (JP); Kazuo Yamada, Sagamico-machi (JP)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/382,590

(22) Filed: Mar. 7, 2003

(30) Foreign Application Priority Data
Mar. 7, 2002 (JP) 2002-062230
Jun. 3, 2002 (JP) 2002-161236
Jun. 25, 2002 (JP) 2002-184330
Sep. 10, 2002 (JP) 2002-263854
Oct. 14, 2002 (JP) 2002-300049
Dec. 20, 2002 (JP) 2002-370104

(51) Int. Cl. A61M 5/00

(52) U.S. Cl. 604/247; 251/149.1; 251/149.9

(57) ABSTRACT

A first object of the invention is to provide a medical device capable of effectively preventing backwards flow of blood.

A first aspect of the invention is a medical device including a check valve therein, the check valve including: a valve holder that has a tapered pipe, and a small diameter pipe connecting to a small diameter opening of the tapered pipe and extending outwards, a larger diameter opening of the tapered pipe being fluid-tightly fitted to an inner surface of a medical device; and a flexible valve body that is fitted to an outer peripheral surface of the small diameter pipe of the valve holder, and has a slit that is opened by fluid pressure from a large diameter opening side of the tapered pipe, or by a lateral pressure, but closed by a fluid pressure from a small diameter pipe side.

### BACKGROUND OF THE INVENTION

1. Field of the Invention

The present invention relates to a medical device for humans or animals and, more particularly, to an outer needle (an indwelling needle) for a catheter, an inner needle, a plug, a wing needle, an infusion tube, or a side branch pipe.

2. Description of the Related Art

The First Prior Art (an Outer Needle for a Catheter and an Inner Needle)

FIG. 30 is an assembly drawing of a structure of a conventional catheter. This catheter includes an indwelling needle (or an outer needle) and an inner needle. The outer needle for a catheter 1 includes a cylindrical hub 11 made of transparent resin, a needle 12 fitted and connected to one end of the hub 11 and made of flexible resin, and a caulking cap 33 made of metal that is fitted into the hub 11 and keeps a fitting connection between the hub 11 and the needle 12 airtightly and fluid-tightly.

The catheter is inserted into a patient's skin for use according to the following procedure. The needle 12 of the outer needle 1 is flexible and elastic, and it is impossible to insert the needle 12 alone into the skin. Thus, a previously prepared inner needle 4 made of metal is inserted into the needle 12 to control flexure of the needle 12. A tip 41 of a needle 42 of the inner needle 4 is protruded beyond a tip of the needle 12, the tip 41 of the needle 42 is inserted into the skin, the tip of the needle 12 is inserted into a desired position on the skin, and then the inner needle 4 only is drawn to keep the outer needle 1 inserted into the skin. Next, an unshown infusion tube is connected into the hub 11 to infuse an infusion solution into a patient's body. At this time, the other end of the infusion tube is, of course, connected to an infusion solution bag.

As described above, in use of the catheter, the inner needle is used as auxiliary means for assisting penetration of the outer needle made of resin into the skin, the outer needle is inserted into the skin together with the inner needle, and the inner needle is drawn after the outer needle penetrates the skin, then the outer needle is connected to the infusion tube. However, depending on blood pressures of the patient, blood in the body flows backwards through the inner needle 4 into the hub 11 of the outer needle 1, and into the infusion tube. If the patient sees this phenomenon through the transparent hub 11 of the outer needle 1, the patient will be surprised and often feel uneasy. Further, the blood flows backwards during connection to the infusion tube, a doctor or a nursing person has to work quickly. Slow work may cause the blood to escape from the hub 11. Hasty work may cause the doctor or the nursing person to stick the drawn needle to his/her finger, leading to developing infection diseases caused by viruses in the blood.

The Second Prior Art (a Wing Needle)

FIG. 31 is a schematic view of a conventional wing needle catheter.

The wing needle catheter 5 includes a needle 52 formed of a metal thin pipe and having a sharp tip 51, a small diameter hub 55 connected to the other end 53 of the needle 52, a flexible tube 57 connected to the other end 56 of the small diameter hub 55, a connection 59 connected to the other end 58 of the flexible tube 57, and two flexible and elastic wings 54a, 54b placed on side surfaces of the small diameter hub 55 and extending to the right and the left, respectively.

An unshown infusion tube can be connected to the connection 59, and for this purpose, a cap 59a attached to the connection 59 is detached, and then an end of the infusion tube is inserted for connection.

Next, the metal needle of the wing needle is kept inserted into a patient's skin, and then the infusion tube is connected into the connection 59 to infuse an infusion solution into a patient's body. At this time, the other end of the infusion tube is, of course, connected to an infusion bag.

The problems of the outer needle for a catheter and the inner needle also apply to the wing needle.

The outer needle for a catheter or the wing needle is kept inserted into a patient's arm for three or four days, and an extension tube is connected to a rear end of the outer needle or the wing needle. In infusion, the infusion bag is connected to the other end of the extension tube, and in other cases, a cap or the like is attached to the other end of the extension tube to temporarily fasten the extension tube on the patient's arm. However, the extension tube may be drawn, or the cap may be detached. Such an accident may cause blood to escape through the outer needle or the wing needle, leading to pollution of pajamas or a bed. If the patient is left for long hours without noticing the accident, there is a risk of deadly loss of blood.

The Third Prior Art (a Medical Plug)

FIG. 32 is a schematic view of an assembly of a conventional inner needle for a catheter and a conventional plug for a catheter connected together.

An inner needle 4 includes a needle 42 formed of a metal thin pipe and having a sharp tip 41, and a hub 43 connected to the other end of the needle 42 and made of transparent resin. The hub 43 is cylindrical, and its free end may be tightly fitted to a small diameter portion 61 of a frame 6 of the plug for a catheter for connection.

The frame 6 of the plug for a catheter is made of transparent resin, and is cylindrical. The plug for a catheter 6 includes no structure therein, and a filter 690 made of a porous material is attached to a flange 63.

The plug for a catheter may connect to a rear end of an outer needle (an indwelling needle). A similar plug may connect to a rear end of a wing needle, an end of an infusion tube, or one end of a three-way stopcock.

The problems of the outer needle for a catheter, the inner needle, and the wing needle described above also apply to the medical plug.

### SUMMARY OF THE INVENTION

A first object of the invention is to provide a medical device capable of effectively preventing escape of blood.

The first object is achieved by a first aspect of the invention according to claim 1, specifically, a medical device including a check valve therein, the check valve including: a valve holder that has a tapered pipe, and a small diameter pipe connecting to a small diameter opening of the tapered pipe and extending outwards, a large diameter opening of the tapered pipe being fluid-tightly fitted to an inner surface of a side branch; and a flexible valve body that is fitted to an outer peripheral surface of the small diameter pipe of the valve holder, and has a slit that is opened by a fluid pressure from a large diameter opening side of the tapered pipe, but closed by a fluid pressure from a small diameter pipe side.

The applicant proposes an indwelling needle (an outer needle) for a catheter including a check valve in Japanese Patent Application No. 2002-062230, an inner needle for a catheter and a wing needle having a check valve in Japanese Patent Application No. 2002-161236, and a plug for a catheter including a check valve in Japanese Patent Application No. 2002-200766.

First Aspect of the Invention

FIG. 33 is a schematic view of a plug for a catheter including a check valve according to a first aspect of the invention, and (a) is a front sectional view thereof and (b) is a side view of a valve body.

A frame 6 made of transparent resin includes a small diameter portion 61, a cylindrical body 62, and a flange 63 connected together, and is rotationally symmetric around a central axis 60, and inner pipes are in communication. An inner pipe 64 of the small diameter portion 61 is the thinnest, an inner pipe 66 of the body 62 is thick and has a diameter gradually increasing toward the flange 63, and a tapered inner pipe 65 is placed between the inner pipe 64 of the small diameter portion 61 and the inner pipe of the body 62. A frame of the conventional plug is strong at any portions, and is not deformed if pressed with a finger.

A caulking cap 33 is sometimes referred to as a caulking pin, and includes a hollow conical tapered portion 331, and a straight pipe 332 connecting to a small diameter opening of the tapered portion 331.

The check valve 3 includes a check valve holder made of metal, and a valve body 32 made of silicone rubber, natural rubber, or synthetic rubber. The check valve holder 31 includes a hollow conical tapered portion 311, a straight pipe 312 in the middle connecting to a small diameter opening of the tapered portion, a rounded portion 313, and a straight pipe 314 with small diameter connecting to a small diameter opening of the rounded portion. The valve body 32 includes a hollow straight pipe 322 and a “ruling pen” portion 321 connecting thereto. Like a “ruling pen” for drawing, two flat plates 324, 325 are placed with their long sides contacting each other near a tip of the “ruling pen” portion 321. When a force acting on an outer surface of the valve body 32 (a fluid pressure) is equal to or larger than a force acting on an inner surface (a fluid pressure), the two flat plates 324, 325 tightly contact each other and do not pass any fluid. On the other hand, when the force acting on the inner surface of the valve body 32 (the fluid pressure) is larger than the force acting on the outer surface (the fluid pressure) by a predetermined value or more, the two flat plates 324, 325 are separated to leave a clearance therebetween and open, and a fluid passes through the opening.

When an indwelling needle (an outer needle) for a catheter or a wing needle including the check valve according to the first aspect of the invention is inserted into a patient's vein, the check valve 3 stops backflow of blood. Thus, a small diameter portion 61 side from the check valve 3 of the inner pipe of the small diameter portion 61 and the inner pipe of the body 62 of the frame 6 is filled with the blood. On the other hand, a flange 63 side of the inside of the valve body 32 and the inner pipe of the body 62 of the frame 6 do not contain blood but contains air. A so-called “air pool” is generated behind the check valve. When an infusion tube is connected to the inner pipe of the body 62 of the frame 6, the “air pool” causes air to be mixed into a front part of the infusion solution, which may cause air to be infused into the patient's vessel. Accordingly, air must be removed from the “air pool”.

Further, blood is sometimes collected from a patient in hospital after infusion, and it is convenient for a medical practitioner or a patient to collect blood from an indwelling needle (an outer needle for a catheter) or a wing needle inserted into a vessel. Inserting a new injection needle into the patient to position a needle tip in a vein for each blood collection increases medical practitioner's work and patient's pain. However, the indwelling needle or the wing needle including the conventional check valve according to the first aspect of the invention stops backflow of the blood and thus makes blood collection impossible.

Therefore, a second object of the invention is to provide a medical device including a check valve that allows temporary backflow.

The second object is achieved by a second aspect of the invention according to claim 2, specifically, a medical device including: a flexible portion, and a check valve in the flexible portion, the check valve including: a valve holder that has a tapered pipe, and a small diameter pipe connecting to a small diameter opening of the tapered pipe and extending outwards, a large diameter opening of the tapered pipe being fluid-tightly fitted to an inner surface of a side branch; and a flexible valve body that is fitted to an outer peripheral surface of the small diameter pipe of the valve holder, and has a slit that is opened by a fluid pressure from a large diameter opening side of the tapered pipe, or by a lateral pressure, but closed by a fluid pressure from a small diameter pipe side, the flexible portion being pressed to press the check valve perpendicularly to a central axis, thus the check valve being deformed and opened.

Second Aspect of the Invention

The applicant proposes an outer needle for a catheter, an inner needle, a wing needle, a plug, or the like having a flexible portion at part of a frame and a check valve in the flexible portion in Japanese Patent Application No. 2002-263854.

FIG. 34 is a front sectional view of a plug for a catheter according to the second aspect of the invention, and FIG. 35 is a plan view of the plug for a catheter according to the second aspect of the invention.

A frame 6 includes a small diameter portion 61, a body 62, and a flange 63, and is made of transparent or translucent soft resin. A flange side of the body 62 is cylindrical, and a small diameter portion side of the body 62 is flat at a bottom 68 and has a thin portion 66 and a button 67 at a top. When a top surface of the button 67 is pressed with a finger, the bottom 68 stably contacts a skin of a patient's arm or thigh, and the thin portion 66 is dented and moves downwards with the frame 6 being not easily moved. Thus, a bottom surface of the thin portion 66 contacts the valve body 32 of the check valve 3 to be pressed downwards.

Preferably, the valve body is made of silicone rubber, urethane rubber, or plastic soft materials, and when a force is applied to the valve body to be elastically deformed, and then the force is removed by releasing the finger, the valve body returns to its original shape. A preferable elastic material has repeatability that allows returning to the original shape even after repeated application and removal of a force.

In the second aspect of the invention, the valve body 32 is in the shape of a “ruling pen” for drawing. Long sides of two flat plates 324, 325 at a tip are placed perpendicularly to the bottom 68 of the frame 6. Usually, the long sides of the flat plates 324, 325 tightly contact each other. However, when the button 67 is pressed with a finger, the thin portion 66 is flexed to press the valve body 32 downwards, and the flat plates 324, 325 are flexed to be arcuate to leave a clearance between the flat plates 324, 325. Therefore, a fluid passes through the check valve 3. On the other hand, when pressing the button 67 is stopped, the valve body 32 returns to its original shape, the flat plates 324, 325 tightly contact each other, and the fluid cannot pass therebetween.

However, in order to keep the check valve open for long hours, a medical practitioner must stay near the frame and continue pressing a protrusion of the plug according to the second aspect of the invention with his/her finger, which is a heavy burden and is inconvenient.

Therefore, a third object of the invention is to provide a medical device that facilitates keeping a check valve open for long hours.

The third object is achieved by a third aspect of the invention according to claim 3, specifically, the medical device according to claim 2, further including a pressing tool capable of keeping the flexible portion pressed and releasing the press.

In a preferable embodiment according to the third aspect of the invention, as described in claim 4, the medical device further includes: a protrusion on the flexible portion; guide grooves extending longitudinally on an outer surface; and a pressing tool with a “quasi-horseshoe-shaped” section that has a right side plate and a left side plate extending downwards from both ends of a central plate toward the same side, and part of the right side plate and part of the left side plate, respectively of the pressing tool engage the guide grooves, the pressing tool slides along a length of the medical device, and when the pressing tool is positioned at and around one end, an inner surface of the central plate of the pressing tool presses the protrusion to open a check valve, and when the pressing tool is positioned at and around the other end, the pressing tool does not press the protrusion.

In another preferable embodiment according to the third aspect of the invention, as described in claim 5, the medical device further includes: a protrusion on the flexible portion; guide grooves extending circumferentially on an outer surface; and an annular pressing tool having a protrusion key on part of an inner surface, and the pressing tool engages the guide groove and is rotatable along the guide groove, and when the pressing tool is rotated to place the protrusion key on the protrusion of the flexible portion, the protrusion is pressed to open the check valve, and when the pressing tool is reversely rotated to separate the protrusion key from the protrusion of the flexible portion, the protrusion is not pressed.

In a further preferable embodiment according to the third aspect of the invention, as described in claim 6, the pressing tool includes a hollow cylinder and a cap, the hollow cylinder is threaded and provided near the flexible portion, the cap is threaded and threads into the hollow cylinder, and has a protrusion for pressing the flexible portion on a bottom surface of the cap, when the cap is rotated, the cap is lowered to cause the protrusion to press the flexible portion to open the check valve, and when the cap is rotated in a reverse direction, the cap does not press the flexible portion.

In a further preferable embodiment according to the third aspect of the invention, as described in claim 7, the medical device further includes a protrusion on the flexible portion, the pressing tool includes an elastic belt and engaging tools, one end of the elastic belt is fastened near the protrusion, one of the engaging tools is mounted to the other end of the elastic belt, the other of the engaging tools is mounted to an outer surface of the medical device on a line connecting a fastening portion and the protrusion at one end of the elastic belt and on a side opposite the fastening portion seen from the protrusion, and when the engaging tool at the other end of the elastic belt and the engaging tool at a frame are engaged, the elastic belt presses the protrusion to open the check valve.

The Fourth Prior Art (a Three-Way Stopcock)

Three-way stopcocks are used for blood transfusion or infusion in hospitals, clinics, or animal hospitals. A three-way stopcock includes: a body having a first branch pipe, a second branch pipe, and a third branch pipe; and a path switching portion that can be rotatably and fluid-tightly fitted to the body, and has a fluid path that provides communication between predetermined branch pipes among said branch pipes, and the path switching portion is rotated with respect to the body to switch the fluid path.

[Patent document 1]

Japanese Patent Laid-Open No. 2002-153562

[Patent document 2]

Japanese Utility Model Registration No. 2599493

[Patent document 3]

Japanese Patent No.

2764432

[Patent document 4]

Japanese Patent Laid-Open No. 10-234863

[Patent document 5]

Japanese Patent Laid-Open No. 9-280394

[Patent document 6]

Japanese Patent Laid-Open No. 8-155039

[Patent document 7]

Japanese Utility Model Laid-Open No. 6-044554

For example, an indwelling needle is inserted into a patient's vein, and a rear end of the indwelling needle and the first branch pipe are connected by a tube. The second branch pipe is connected to a blood transfusion bag by another tube, and the third branch pipe is connected to a syringe containing an inhibitor of blood coagulation by a further tube. With the path switching portion being rotated to provide communication between the first, the second, and the third branch pipes, blood and the inhibitor of blood coagulation are infused into the patient at the same time. After a predetermined time, with the path switching portion being further rotated to provide communication between the first and the second branch pipes, the blood only is infused into the patient. In this way, the path switching portion is appropriately rotated for blood transfusion.

An infusion solution, a glucose solution, an antibiotic solution, or the like are switched and mixed every predetermined hour for infusion into the patient's vein. If required, the paths switching portion is appropriately rotated to collect blood in midstream.

However, when the three-way stopcock is used, a medical practitioner such as a doctor or a nursing person, or a patient may rotate the paths switching portion to switch the path of the branch pipe to a wrong position, leading to a medical accident.

The patient (especially, an infant or a demented elderly) may rotate the path switching portion, and thus the patient' condition may sharply become worse and the patient may fall in danger of life.

Therefore, a fourth object of the invention is to prevent medical accidents or accidents resulting from unintentional operations that may occur in using the three-way stopcock, and for this purpose, to develop a novel side branch pipe.

The above object is achieved by a fourth aspect of the invention according to claim 10, specifically, a medical side branch pipe in which a first straight pipe and a second straight pipe are arranged on a line, and one or more side branches intersect the first straight pipe and the second straight pipe at a confluence and communicate with each other, wherein at least one side branch has a check valve therein such that a fluid and a gas from a free end of the side branch to the confluence pass, but a fluid and a gas from the confluence to the free end are blocked and do not flow.

In a preferable embodiment according to the fourth aspect of the invention, as described in claim 11, there is provided a medical side branch pipe in which a first straight pipe and a second straight pipe are arranged on a line, and one or more side branches intersect the first straight pipe and the second straight pipe at a confluence and communicate with each other, wherein at least one side branch has a check valve therein, part of a side surface of the side branch near the check valve is a flexible portion made of an elastic material, and when the flexible portion is not pressed, a fluid and a gas from a free end of the side branch pipe to the confluence pass, but a fluid and a gas from the confluence to the free end are blocked and do not flow, while when the flexible portion is pressed, the fluid and the gas pass from the confluence to the free end.

In a preferable embodiment according to the fourth aspect of the invention, as described in claim 12, the medical side branch pipe further includes a pressing tool capable of keeping the flexible portion pressed and releasing the press.

In a preferable embodiment according to the fourth aspect of the invention, as described in claim 13, the check valve includes: a valve holder that has a tapered pipe, and a small diameter pipe connecting to a small diameter opening of the tapered pipe and extending outwards, a large diameter opening of the tapered pipe being fluid-tightly fitted to an inner surface of a side branch; and a flexible valve body that is fitted to an outer peripheral surface of the small diameter pipe of the valve holder, and has a slit that is opened by a fluid pressure from a large diameter opening side of the tapered pipe, or by a lateral pressure, but closed by a fluid pressure from a small diameter pipe side.

In the second, the third, and the fourth aspects of the invention, no mark is provided that indicates a position to be pressed with a finger or a position of the pressing tool, and it is difficult to find or confirm such positions. Especially, a light is turned off or darkened during the night, thus the medical practitioner such as the nursing person turns on a flashlight to find or confirm the positions, and such work is difficult.

Therefore, a fifth object of the invention is to provide a medical device that facilitates finding and confirming a position to be pressed with a finger or a position of a pressing tool.

The fifth object is achieved by a fifth aspect of the invention according to claim 18, specifically, the medical side branch pipe according to any one of claims 2 to 7, 11 and 12, wherein the flexible portion is colored, or coated with fluorescent paint, or a color tape or a fluorescent paint tape is affixed to the flexible portion.

The fifth object is also achieved by a sixth aspect of the invention according to claim 19, specifically, the medical device according to any one of claims 3 to 7, or claim 12, wherein a material for the pressing tool contains fluorescent paint, or the pressing tool is colored, or coated with fluorescent paint, or a color tape, a fluorescent paint tape, or a reflection sheet is affixed to the pressing tool.

### BRIEF DESCRIPTION OF THE DRAWINGS

FIG. 1 is a schematic sectional view of an indwelling needle according to a first embodiment;

FIG. 2 is a schematic sectional view of an indwelling needle according to a second embodiment;

FIG. 3 is an assembly drawing of an inner needle and an outer needle for a catheter according to a third embodiment;

FIG. 4 is a sectional view of essential portions and shows a check valve in the inner needle for the catheter according to the third embodiment;

FIG. 5 is a sectional view of essential portions and shows a check valve in the outer needle of the catheter according to the third embodiment;

FIG. 6 shows a fourth embodiment of a check valve mounted into an inner needle hub;

FIG. 7 is a schematic view of a plug for a catheter according to a fifth embodiment, and (a) is a front sectional view thereof and (b) is a side view of a valve body;

FIG. 8 is a schematic view of a plug for a catheter according to a sixth embodiment, and (a) is a front sectional view thereof and (b) is a side view of a valve body;

FIG. 9 is a schematic view of a plug for a catheter according to a seventh embodiment, and (a) is a sectional view thereof and (b) is a side view of a valve body;

FIG. 10 is a schematic view of a plug for a catheter according to an eighth embodiment, and (a) is a sectional view thereof and (b) is a side view of a valve body;

FIG. 11 is a front sectional view of a plug for a catheter according to an embodiment of a medical device of a second aspect of the invention;

FIG. 12 is a plan view of the plug for a catheter according to the embodiment of the medical device of the second aspect of the invention;

FIG. 13 is a sectional view along the line A-A in FIG. 11;

FIG. 14 illustrates an operation of a check valve included in the plug for a catheter according to the embodiment;

FIG. 15 illustrates a plug for a catheter according to an embodiment of a third aspect of the invention, and (a) is a front sectional view, (b) is a sectional view along the line A-A in (a), and (c) is a sectional view along the line B-B in (a);

FIG. 16 illustrates a pressing tool of a plug for a catheter according to an embodiment of the third aspect of the invention, and (a) is a front view thereof and (b) is a side view thereof;

FIG. 17 illustrates an operation of the plug for a catheter of the third aspect of the invention;

FIG. 18 illustrates an operation of the plug for a catheter of the third aspect of the invention;

FIG. 19 is a plan view of a side branch pipe according to a seventeenth embodiment;

FIG. 20 is a plan view of a side branch pipe according to an eighteenth embodiment;

FIG. 21 is a front view of the side branch pipe according to the eighteenth embodiment;

FIG. 22 is a plan view of a side branch pipe according to a nineteenth embodiment;

FIG. 23 is a plan view of a side branch pipe according to a twentieth embodiment;

FIG. 24 is a front view of the side branch pipe according to the twentieth embodiment;

FIG. 25 is a plan view of a side branch pipe according to a twenty-first embodiment;

FIG. 26 is a front view of the side branch pipe according to a twenty-first embodiment;

FIG. 27 is a schematic view of a modification according to the twenty-first embodiment;

FIG. 28 is a plan view of a side branch pipe according to a twenty-second embodiment;

FIG. 29 is a front view of the side branch pipe according to the twenty-second embodiment;

FIG. 30 is an assembly drawing of a structure of a conventional catheter;

FIG. 31 is a schematic view of a conventional wing needle catheter;

FIG. 32 is a schematic view of an assembly of a conventional inner needle and a plug for a catheter connected to each other;

FIG. 33 is a schematic view of a plug for a catheter including a check valve of a first aspect of the invention, and (a) is a front sectional view thereof and (b) is a side view of a valve body;

FIG. 34 is a front sectional view of a plug for a catheter according to the second aspect of the invention;

FIG. 35 is a plan view of a plug for a catheter according to a twenty-third embodiment of the invention;

FIG. 36 is a plan view of a medical side branch pipe according to a twenty-fourth embodiment of a fifth aspect of the invention;

FIG. 37 is a front views of a plug for a catheter according to a twenty-fifth embodiment of a fifth aspect of the invention; and

FIG. 38 is a front view of a plug for a catheter according to a twenty-fifth embodiment of a fifth aspect of the invention.

FIG. 39 is a schematic view of an indwelling needle according to a modification of the second embodiment of a first aspect of the invention.

### DETAILED DESCRIPTION OF THE PREFERRED EMBODIMENTS

Hereinafter, embodiments of the invention will be described in detail with reference to the accompanying drawings.

First Embodiment (First Aspect of the Invention)

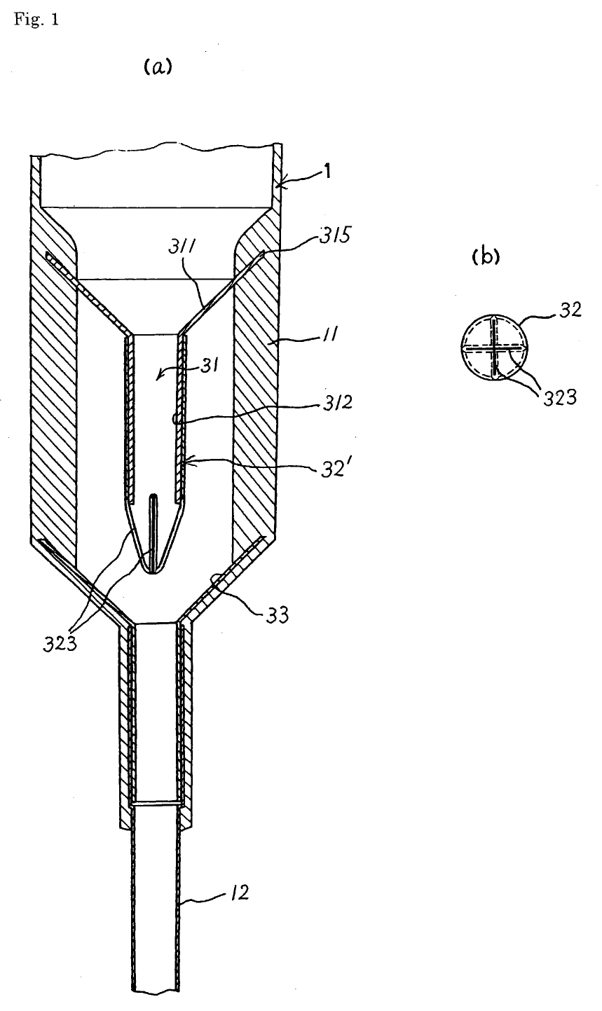

FIG. 1 is a schematic sectional view of an indwelling needle according to a first embodiment. The first embodiment provides an indwelling needle having a hub 11 and a needle portion 12 of an outer needle 1 integrated together via a caulking cap 33, wherein at a position near the needle portion 12 in the hub 11, an opening edge 315 of a tapered pipe of a check valve holder 31 that includes a tapered pipe 311 having a diameter gradually increasing upwards, and a straight pipe 312 connecting to a small diameter opening of the tapered pipe 311 and extending along a pipe axis, integrated together is insert molded airtightly or fluid-tightly to be integral with a thick portion formed on an inner peripheral surface of the hub 11 by insert molding means for resin molding of the hub 11.

Further, a cylindrical valve 32′ is adhered and fastened to the straight pipe 312, the cylindrical valve 32′ being capable of blocking a lower opening of the straight pipe 312. The cylindrical valve 32 is made of flexible and elastic rubber or resin, and a lower end thereof has a bottom with substantially v-shaped sides. The bottom has a cross-shaped valve slit 323. Besides four parts, the valve slit may be divided into two parts, three parts, or six parts. The valve slit 323 can be molded when molding the cylindrical valve 32′, and is usually blocked by the action of flexibility and elasticity so as not to pass any fluid, but when a fluid pressure from above acts on the valve slit 323, the fluid pressure opens the valve slit 323 to allow the fluid to pass. Of course, the cylindrical valve 32′ must be made of a drug-resistant material such that an infusion solution does not change the quality of the material.

Thus, in use of the cylindrical valve 32′ of this structure, the valve slit 323 is usually blocked, thus a blood pressure from a needle portion 12 side does not open the valve slit 323, effectively preventing upward backflow of blood from the hub 11. When a fluid pressure of an infusion solution falling from a hub 11 side, the fluid pressure opens the valve slit 323 to allow a fluid to be supplied toward the needle portion 12.

Second Embodiment (First Aspect of the Invention)

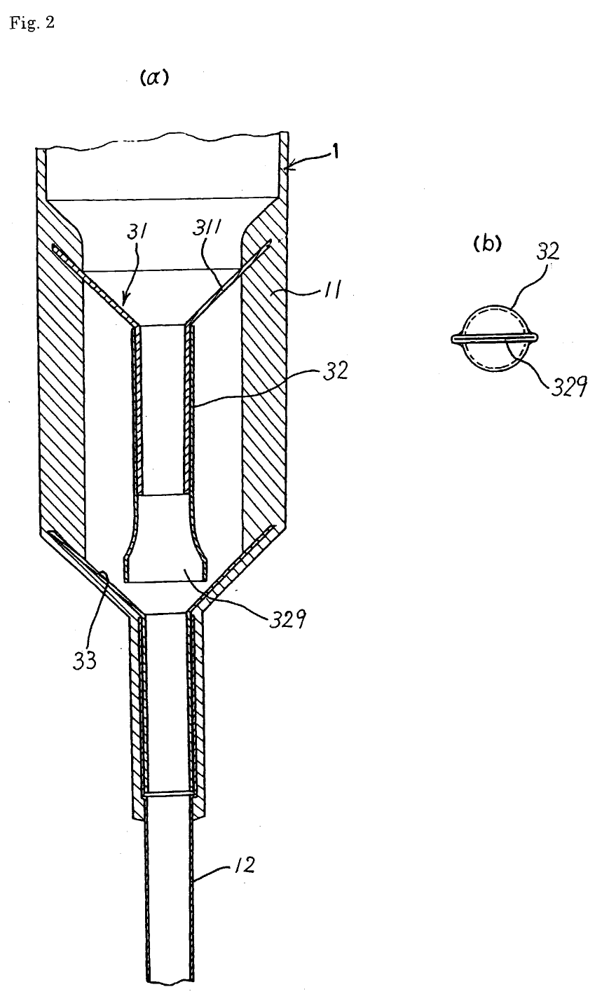

FIG. 2 is a schematic sectional view of an indwelling needle according to a second embodiment. The second embodiment is a modification of the cylindrical valve 32′ in the first embodiment, and the lower end opening of the cylindrical valve 32′ is pressed from both sides to form an abutting valve slit 329 in a flat shape. The abutting valve slit 329 can be molded using an injection molding mold having a relatively simple structure, like molding of the cylindrical valve 32′ made of flexible and elastic resin or rubber, thus achieving greater production economy of the cylindrical valve 32′ with the abutting valve slit 329.

According to the indwelling needle (the outer needle for a catheter) in the first and the second embodiments of the first aspect of the invention, the indwelling needle includes the check valve, and thus allows effective prevention of backflow of blood into the indwelling needle which tends to be caused by a negative pressure in inserting the outer needle into a skin together with an inner needle and then drawing the inner needle from the outer needle, and by a blood pressure in a vein that is reached by the insertion.

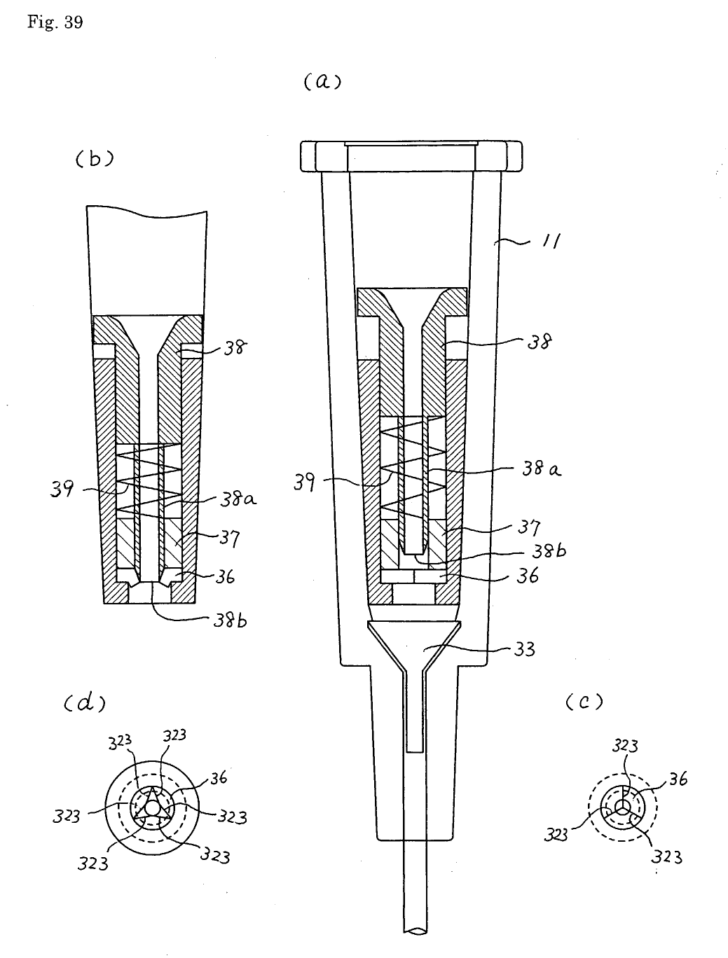

FIG. 39 is a schematic view of an indwelling needle according to a modification of the second embodiment of a first aspect of the invention. A disc valve 36 with valve slits 323 is positioned near a larger diameter opening of a caulking cap 33. A guide pipe 37 is contacted on the disc valve 36 with one end of the guide pipe. An extrusion part 38a of a needle guide 38 is inserted into the inner of the guide pipe. The needle guide 38 is guided by the guide pipe 37 and can be moved along the axis of a hub 11. The needle guide is forced to move away from the disc valve 36 by a spring 39. When the needle guide 38 moves to the disc valve 36 by inserting a inner needle into an indwelling needle against the spring force, the end 38b of the needle guide 38 pushes the disc valve 36 and the disc valve 36 is opened as shown in FIGS. 39(b) and (d). On the other hand, when the inner needle is drawn from the indwelling needle, the needle guide moves away from the disc valve by spring force and the disc is closed as shown in FIGS. 39(a) and (c).

Third Embodiment (First Aspect of the Invention)

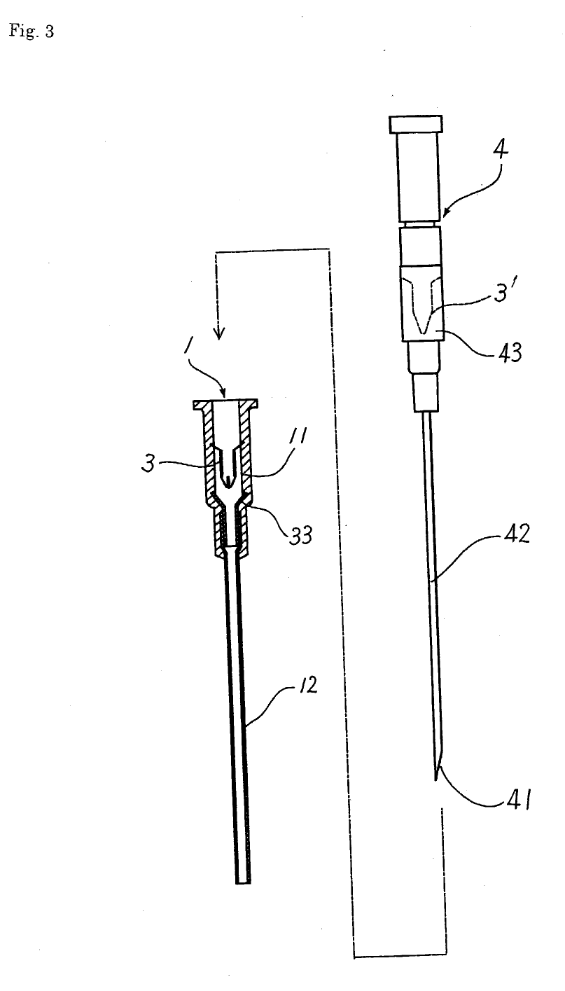

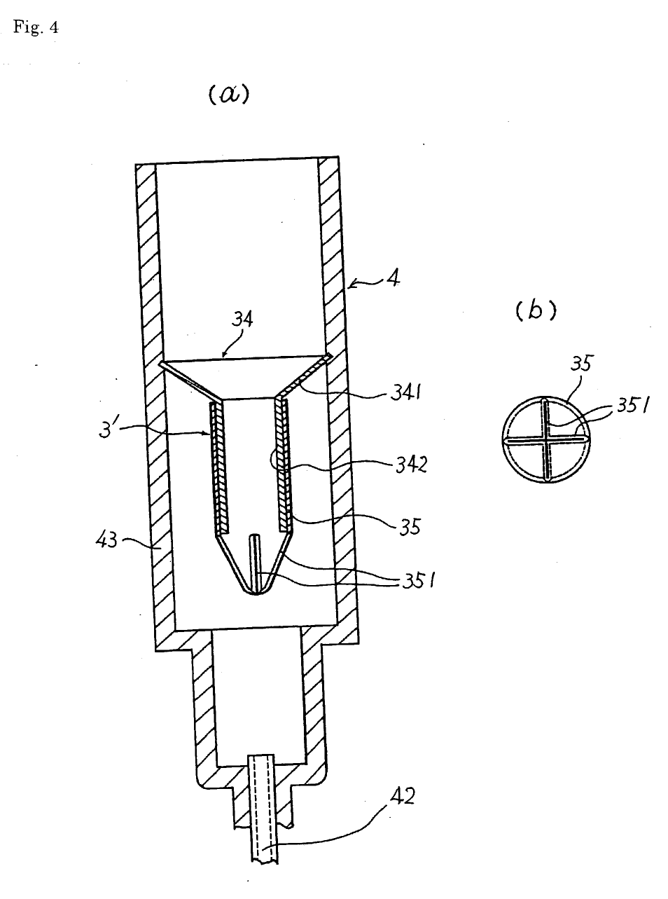

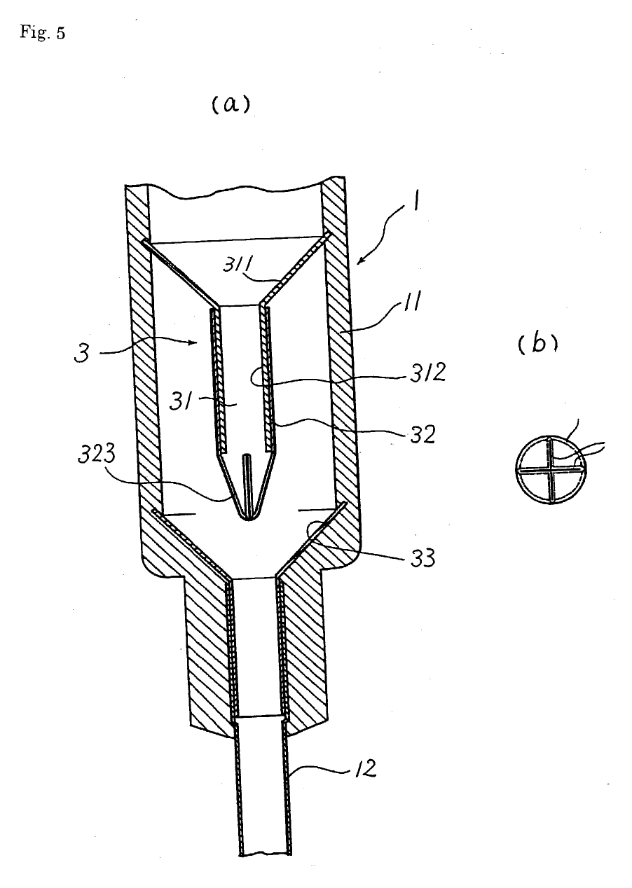

FIG. 3 is an assembly drawing of an inner needle and an outer needle for a catheter according to this embodiment, FIG. 4 is a sectional view of essential portions and shows a check valve in the inner needle for the catheter according to this embodiment, and FIG. 5 is a sectional view of essential portions and shows a check valve in the outer needle for the catheter according to this embodiment.

Reference numeral 4 denotes an inner needle for a catheter, and reference numeral 1 denotes an outer needle for a catheter. Like a conventional example, the inner needle for a catheter 4 is used as an auxiliary tool for inserting the outer needle for a catheter 1 into a skin.

The inner needle for a catheter 4 is formed of a metal thin pipe, and includes a needle portion 42 having a sharp tip 41 to be inserted into the skin, and a transparent hub 43 made of resin connected to the other end of the needle portion 42 fluid-tightly. The hub 43 includes a check valve 3′ shown in FIG. 4. The transparent hub 43 allows backflow of blood into the hub to be visible, and allows indirect confirmation of whether the tip of the inner needle for a catheter 4 is positioned in a vessel.

The check valve 3′ includes a valve holder 34 and a valve body 35, and the valve holder 34 is substantially in a funnel shape, and includes a tapered pipe 341 having a diameter gradually increasing upwards, and a small diameter pipe 342 connecting to an outer side of a small diameter opening of the tapered pipe 341 and extending along a pipe axis, integrated together. The valve body 35 tightly contacts an outer peripheral surface of the small diameter pipe 342 and is supported so as to block an opening end of the small diameter pipe 342 of the valve holder 34, and is cylindrical and made of flexible and elastic rubber or resin.

A lower end of the valve body 35 has a cross-shaped valve slit 351. Besides four parts of the cross, the valve slit 351 may be divided into two parts, three parts, or six parts. The valve slit 351 can be molded when molding the valve body 35, and is usually blocked by the action of flexibility and elasticity so as not to pass any fluid, but when a fluid pressure from a hub side acts on the valve slit 351, the fluid pressure opens the valve slit 351 to allow the fluid to pass. Of course, a material for the valve body 35 must be made of a drug-resistant material such that an infusion solution does not change the quality of the material.

For the tapered pipe 341 of the check valve 3′ thus configured, a large diameter side edge of the tapered pipe 341 and the hub 43 are connected fluid-tightly or airtightly by insert molding when resin molding the hub 43 of the inner needle for a catheter 4, and the needle portion 42 is attached to a lower end of the hub 43, thus completing the inner needle for a catheter 4.

Next, a configuration of the outer needle for a catheter 1 used together with the inner needle for a catheter 4 will be described. The outer needle for a catheter 1 includes a hub 11 made of resin, and a needle portion 12 connected to one end of the hub 11 via a caulking cap 33 fluid-tightly and airtightly and made of, for example, silicone rubber.

The hub 11 includes a check valve 3 like a check valve 3′ in the inner needle for a catheter 4. Specifically, as shown in FIG. 5, the check valve 3 includes a valve holder 31 in a funnel shape attached to an inner peripheral surface of the hub 11 fluid-tightly and airtightly, and a cylindrical valve body 32 made of a flexible and elastic material and connected to an outer peripheral surface of a straight pipe 312 of the valve holder 31 fluid-tightly and airtightly. At a lower end of the valve body 32, a valve slit 323 is provided that is usually blocked by the action of flexibility and elasticity so as not to pass any fluid, and when a fluid pressure from a hub side acts on the valve slit 323, the valve slit 323 opens to allow the fluid to pass.

Configurations of the check valves 3′ and 3, placed in the inner needle for a catheter 4 and the outer needle for a catheter 1 are not limited to the above described configurations, but any configurations that prevent a fluid from passing in one direction may be applied.

When the inner needle for a catheter 4 and the outer needle for a catheter 1 having the above described configurations are used for infusion into a patient, the needle portion 42 of the inner needle for a catheter 4 including the check valve 3′ is first inserted into the outer needle for a catheter 1 from an opening side of the hub 11 of the outer needle for a catheter 1 to penetrate the check valve 3 and the needle 1 so that the tip 41 of the needle portion 42 protrudes beyond the tip of the needle portion 12.

Then, the tip of the needle portion 12 of the outer needle for a catheter 1 is inserted into the patient's skin together with the tip 41 of the needle portion 42, and the tip of the needle portion 12 and the tip 41 of the inner needle 4 are inserted into a desired position on the skin. At this time, a blood pressure in a body may sometimes cause blood to flow backwards into the needle portion 42, but the backflow of the blood is blocked by a valve action of the check valve 3′ in the inner needle for a catheter 4, thus preventing the blood from escaping outside the inner needle for a catheter 4.

Next, the inner needle for a catheter 4 is drawn from the outer needle for a catheter 1 for connecting an unshown infusion tube to the hub 11 of the outer needle for a catheter 1. At this time, blood may flow backwards into the needle portion 12, but the backflow of the blood is blocked by the action of the check valve 3 in the hub 11 of the outer needle for a catheter 1, thus effectively preventing the blood from escaping outside the outer needle for a catheter 1.

Then, a tip of the unshown infusion solution tube is connected to an infusion bag, which is mounted to a higher place, to start infusion. A fluid pressure of the infusion solution caused by a difference in height between the infusion bag and the outer needle for a catheter 1 opens the check valve 3 to supply the infusion solution into the body through the check valve 3.

Therefore, according to the third embodiment, the inner needle for a catheter 4 includes the check valve 3′, thus even if the blood flows backwards into the inner needle for a catheter 4 when inserting the inner needle for a catheter 4 into the skin, the check valve 3′ can prevent the blood from escaping outside the inner needle for a catheter 4.

Fourth Embodiment (First Aspect of the Invention)

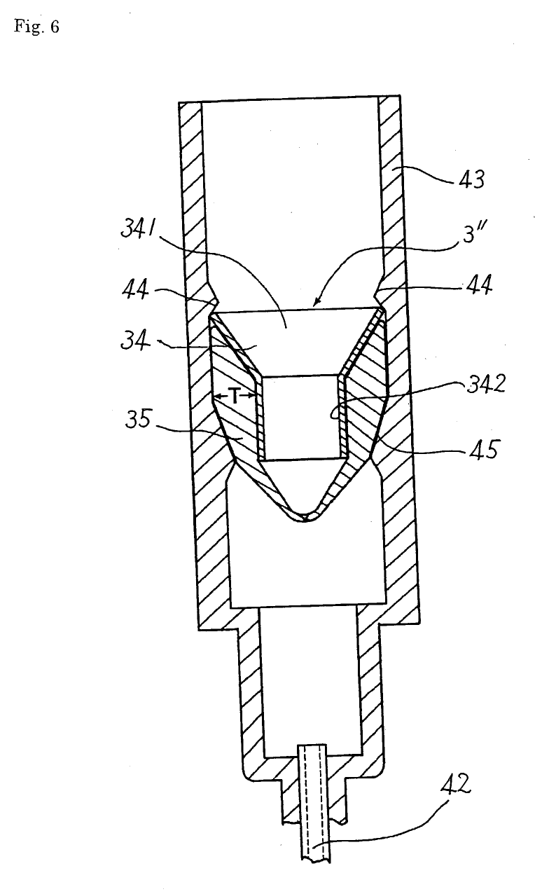

FIG. 6 shows another embodiment (fourth embodiment) of a check valve mounted into an inner needle hub. The check valve 3″ includes a valve holder 34 in a funnel shape engaging a latching step 44 formed on an inner peripheral surface of the hub 43 fluid-tightly or airtightly, and a valve body 35 fitted to and held by an outer peripheral surface of the small diameter pipe 342 of the valve holder 34 and made of a flexible and elastic material.

A periphery of an opening of the valve body 35 has a thickness T and thus can be compressed between a tapered surface 45 formed on the inner peripheral surface of the hub 43 and tapered downwards, and an outer surface of the valve holder 34. A bottom of the valve body 35 has a valve slit that is usually blocked by the action of flexibility and elasticity so as not to pass any fluid.

Therefore, according to the embodiment shown in FIG. 6, the periphery of the opening of the valve body 35 is formed to be thick so as to be compressed and held between the tapered surface 45 formed on the inner surface of the hub 43 and the outer surface of the valve holder 34, thus securing a contact holding force between the valve body 35 and the valve holder 34, eliminating a risk of separation of the valve body 35 from the valve holder 34, and increasing reliability of the structure.

According to the needle for a catheter in the third embodiment of the first aspect of the invention, the needle includes the check valve of the blood, thus the blood does not flow backwards from the needle for a catheter even if an extension tube is drawn or a cap is detached during infusion without the needle inserted into the skin. Therefore, even if the extension tube is drawn or the cap is detached, a bed sheet or pajamas are not polluted with blood, and further, even if a patient is left for long hours without noticing such accidents, no blood escapes, and there is no risk of deadly loss of blood, thus providing safety to the patient.

According to the needle for a catheter in the fourth embodiment of the first aspect of the invention, the valve body is held between the outer surface of the valve holder and the inner surface of the inner needle hub, which prevents the valve body from being separated in the hub, thus increasing reliability of the structure.

Fifth Embodiment (First Aspect of the Invention)

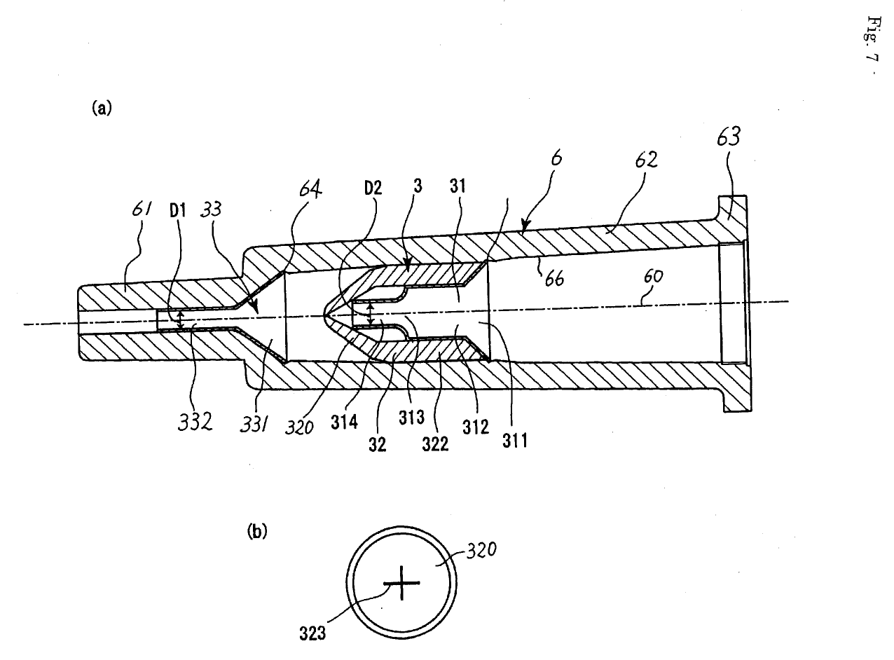

FIG. 7 is a schematic view of a plug for a catheter according to a fifth embodiment, and (a) is a front sectional view thereof and (b) is a side view of a valve body.

The plug for a catheter includes a frame 6 made of transparent resin, a caulking cap 33 made of metal, and a check valve 3.

The frame 6 made of transparent resin includes a small diameter portion 61, a cylindrical body 62, and a flange 63 connected together, and is rotationally symmetric around a central axis 60, and inner pipes are in communication. An inner pipe of the small diameter portion 61 is the thinnest, an inner pipe of the body 62 is thick and has a diameter gradually increasing toward the flange 63, and a tapered pipe is placed between the inner pipe of the small diameter portion 61 and the inner pipe of the body 62.

The caulking cap 33 is sometimes referred to as a caulking pin, and includes a hollow conical tapered portion 331, and a straight pipe 332 connecting to the small diameter opening of the tapered portion 331.

The check valve 3 includes a check valve holder 31 made of metal, and a valve body 32 made of silicone rubber, natural rubber, or synthetic rubber. The check valve holder 31 includes a hollow conical tapered portion 311, a straight pipe 312 connecting to a small diameter opening of the tapered pipe portion 311, a rounded portion 313, and a straight pipe 314 connecting to a small diameter opening of the rounded portion 313. The valve body 32 includes a hollow conical portion 320 and a hollow straight pipe 322 connecting to a large diameter opening of the hollow conical portion 320. A top of the hollow conical portion 320 has a valve slit 323. The valve slit 323 is in a cross shape, and four straight cuts cross at the top at a 90° angle.

The caulking cap 33 is placed in contact with a tapered surface of the frame 6, and an outer edge of a large diameter opening of the caulking cap 33 is fitted and fastened to a circumferential groove 64 on an inner surface of the frame 6.

The check valve 3 is placed in the body 62 of the frame 6, and an outer edge of the large diameter opening of the check valve holder 31 is fitted and fastened to a circumferential groove 65 on an inner surface of the body 62. An inner surface of the hollow straight pipe 322 of the valve body 32 contacts outer surfaces of the tapered pipe portion 311 and the straight pipe 312 of the check valve holder 31 fluid-tightly or airtightly so as to prevent both a fluid and a gas from passing. An outer surface of the hollow straight pipe 322 of the valve body 32 tightly contacts the inner surface of the body 62 of the frame 6 to prevent the check valve 3 from moving with respect to the frame 6 and prevent the valve body 32 from being detached from the check valve holder 31.

Preferably, an inner diameter D1 of the hollow straight pipe 332 of the caulking cap 33 is equal to an inner diameter D2 of the straight pipe 314 of the check valve holder 31. Thus, the inner needle for a catheter can pass through the check valve 3 and the caulking cap 33. At this time, in order to guide a tip of the needle for a catheter near a central axis and prevent damage to the tip, the caulking cap 33 has the tapered portion 331, and the check valve holder 31 has the rounded portion 313.

The valve body 32 has a hollow conical shape, thus when a force acting on the inner surface (a fluid pressure) is larger than a force acting on the outer surface (a fluid pressure) by a predetermined value or more, the valve slit 323 are automatically separated to open. On the other hand, when the force acting on the outer surface (the fluid pressure) is equal to or larger than the force acting on the inner surface (the fluid pressure), the valve slit 323 are not separated and kept closed.

Sixth Embodiment (First Aspect of the Invention)

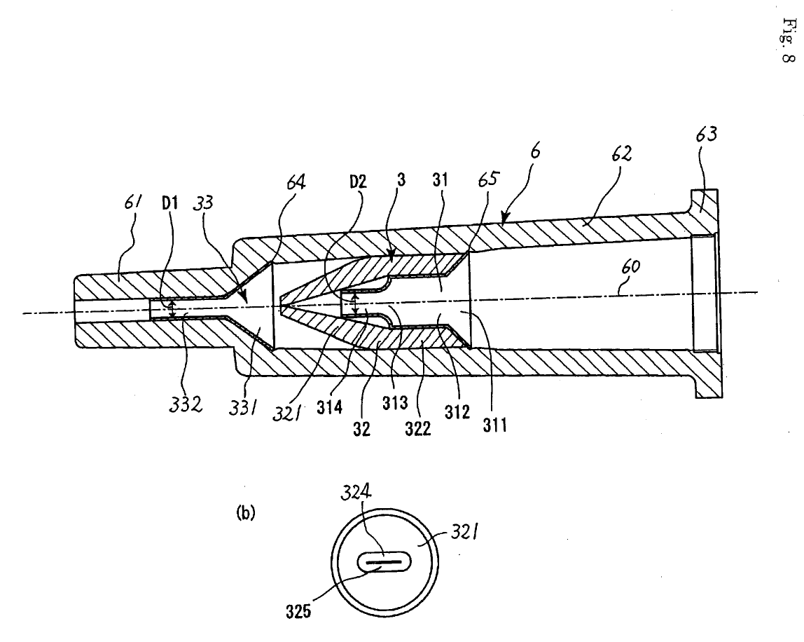

FIG. 8 is a schematic view of a plug for a catheter according to a sixth embodiment, and (a) is a front sectional view thereof and (b) is a side view of a valve body.

The plug for a catheter according to the sixth embodiment includes a frame 6 made of transparent resin, a caulking cap 33 made of stainless steel, and a check valve 3.

The frame 6 made of transparent resin and the caulking cap 33 made of metal are similar to those in the fifth embodiment except for a shape of a valve body 32 of the check valve 3. The valve body 32 includes a hollow straight pipe 322 and a “ruling pen” portion 321 connecting thereto. Like a “ruling pen” for drawing, two flat plates 324, 325 are placed with their long sides contacting each other near a tip of the “ruling pen” portion 321, and the tip has a structure similar to a structure of a tip of the “ruling pen”. When a force acting on an outer surface of the valve body 32 (a fluid pressure) is equal to or larger than a force acting on an inner surface (a fluid pressure), the two flat plates 324, 325 tightly contact each other and do not pass any fluid. On the other hand, when the force acting on the inner surface of the valve body 32 (the fluid pressure) is larger than the force acting on the outer surface (the fluid pressure) by a predetermined value or more, the two flat plates 324, 325 are separated to leave a clearance therebetween and open, and a fluid passes through the opening. Like the fifth embodiment, the valve body in the sixth embodiment must be made of a flexible and elastic material or have a flexible and elastic structure, and the material and the structure must be resistant to drug. For example, the material may include a flexible material such as silicone rubber, natural rubber, or synthetic rubber, and the structure may include a leaf spring structure of a stainless steel sheet or a structure having a rotary axis and a spring such as a clothespin.

Seventh Embodiment (First Aspect of the Invention)

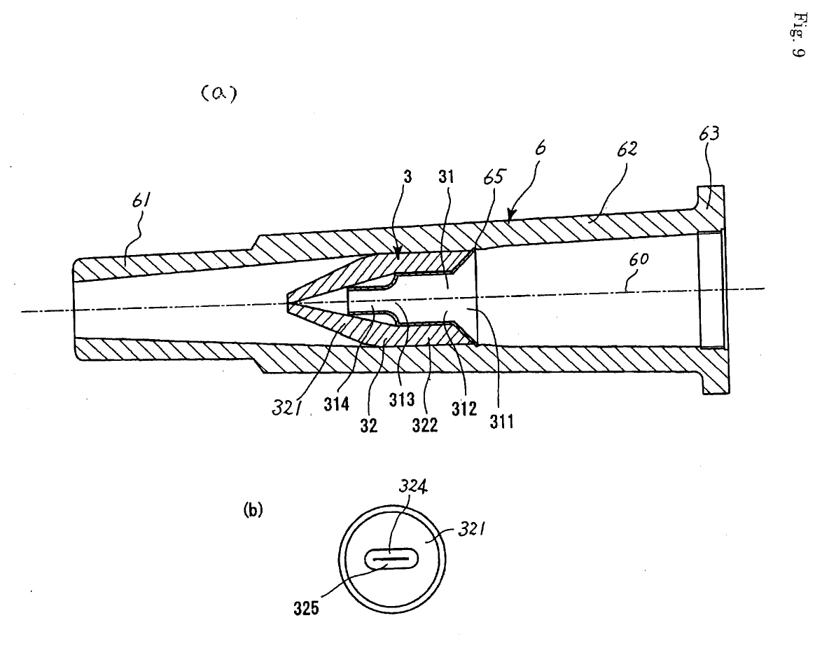

FIG. 9 is a schematic view of a plug for a catheter according to a seventh embodiment, and (a) is a sectional view thereof and (b) is a side view of a valve body.

The plug for a catheter according to the seventh embodiment includes a frame 6 made of transparent resin, and a check valve 3. Unlike the fifth and the sixth embodiments, a caulking cap 33 is not used in the seventh embodiment.

The frame 6 made of transparent resin includes a small diameter portion 61, a cylindrical body 62, and a flange 63 connected together in sequence, and is rotationally symmetric around a central axis 60, and inner pipes are in communication. Diameters of the inner pipes vary along the entire length of the inner pipes, and an end of the small diameter portion 61 has the smallest diameter, and an end of the flange 63 has the largest diameter.

Like the sixth embodiment, the valve body 32 of the check valve 3 includes a hollow straight pipe 322, and a “ruling pen” portion 321 connecting thereto.

Eighth Embodiment (First Aspect of the Invention)

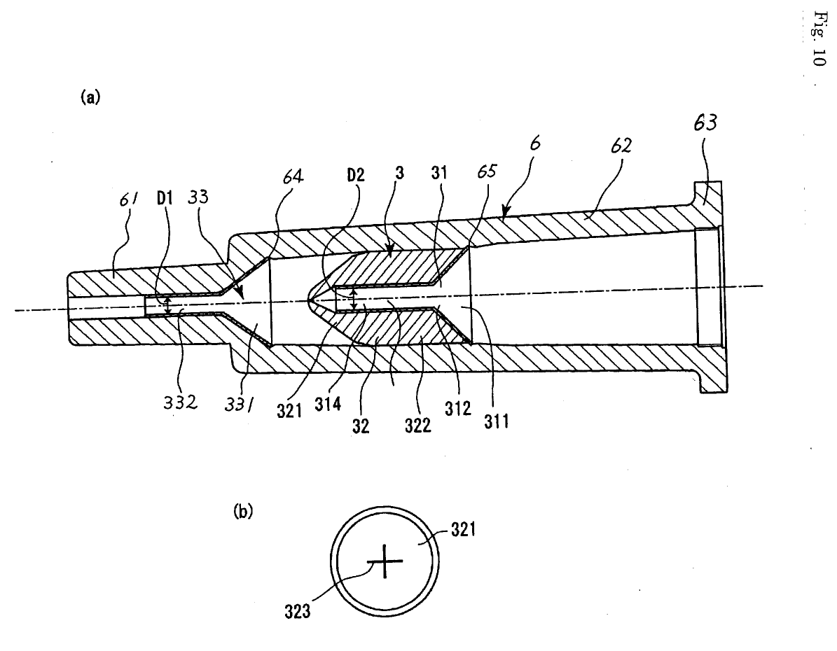

FIG. 10 is a schematic view of a plug for a catheter according to an eighth embodiment, and (a) is a sectional view thereof and (b) is a side view of a valve body.

The plug for a catheter according to the eighth embodiment is substantially the same as the plug for a catheter according to the fifth embodiment expect for a shape of a check valve holder 31, which includes a tapered pipe portion 311, and a straight pipe 316 connecting to a small diameter opening of the tapered portion 311.

The structures and the operations of the plugs for a catheter according to the fifth, the sixth, the seventh, and the eighth embodiments have been described above.

Now, embodiments of use of a plug for a catheter according to the invention will be described.

Ninth Embodiment (First Aspect of the Invention)

A small diameter portion of a plug for a catheter according to the invention can be inserted into a rear end of a frame of an inner needle for a catheter having no backflow preventing device for use without escape of a fluid. When the inner needle for a catheter is inserted into a skin, and a needle tip is place in a vessel such as a vein, a blood pressure causes blood to flow into the inner needle for a catheter, pass through the frame made of transparent resin, reach the plug for a catheter made of transparent resin. Visual check of the blood allows indirect confirmation that the needle tip is positioned in the vessel, and a check valve placed in the plug for a catheter according to the invention prevents backflow of the blood. Thus, there is no risk of loss of blood to provide safety to a patient, and a doctor or a nursing person can work without haste.

Tenth Embodiment (First Aspect of the Invention)

A small diameter portion of a plug for a catheter according to the invention can be inserted into a rear end of a frame of an outer needle (an indwelling needle) for a catheter having a backflow preventing device for use without escape of a fluid. When an inner needle for a catheter is inserted into the outer needle (the indwelling needle) for a catheter, and with a tip of the inner needle protruding beyond an tip of the outer needle, the needle is inserted into a skin of a patient or an animal to reach a vessel, blood flows backwards through the inner needle for a catheter and the outer needle for a catheter. However, the backflow preventing device of the outer needle for a catheter, and a check valve of the plug for a catheter according to the invention that is connected to a rear end of the outer needle for a catheter doubly prevent backflow of blood or a body fluid.

Eleventh Embodiment (First Aspect of the Invention)

A plug for a catheter according to the invention is inserted into a rear end of a wing needle having no backflow preventing device, and an infusion tube is inserted into a flange of the plug for a catheter for use. When a tip of the wing needle is placed in a vessel, a blood pressure causes blood to flow backwards, but a check valve of the plug for a catheter according to the invention prevents the backflow to prevent loss of blood or prevent the blood from flowing into the infusion tube. Especially, even if the infusion tube is detached from the wing needle during infusion, no blood escapes, and a bed sheet is less prone to be polluted with blood. The blood does not flow into the infusion tube, thus the infusion tube and an infusion bag after use can be disposed of as municipal wastes. This reduces processing costs and the amount of industrial wastes to delay developing new landfill sites due to lack of landfill sites.

The plugs for a catheter according to the sixth to the eleventh embodiments of the first aspect of the invention include a backflow preventing mechanism and thus have following advantages. (1) The plug for a catheter according to the invention is connected to the end of the inner needle for a catheter, the outer needle for a catheter, or the hub of the wing needle having no backflow preventing mechanism, thus the check valve in the plug for a catheter prevents backflow of blood or a body fluid caused by a blood pressure. As a result, the patient does not feel uneasy, a bed sheet or pajamas are not polluted with blood, and the doctor or the nursing person can perform medical treatment without haste, thus reducing the number of accidents of sticking the needle to his/her finger or hand. (2) The plug for a catheter according to the invention is connected to the end of the inner needle for a catheter, the outer needle, or the hub of the wing needle having a backflow preventing mechanism, thus the advantage in (1) is further secured, and when the infusion tube is inserted into the rear end of the plug for a catheter, the blood does not flow backwards into the infusion tube, thus the infusion tube after use can be disposed of as a municipal waste. This reduces waste processing costs and the amount of industrial wastes to prolong a life of a landfill site, thereby contributing to the environment.

Next, embodiments of a second aspect of the invention will be described.

Twelfth Embodiment (Second Aspect of the Invention)

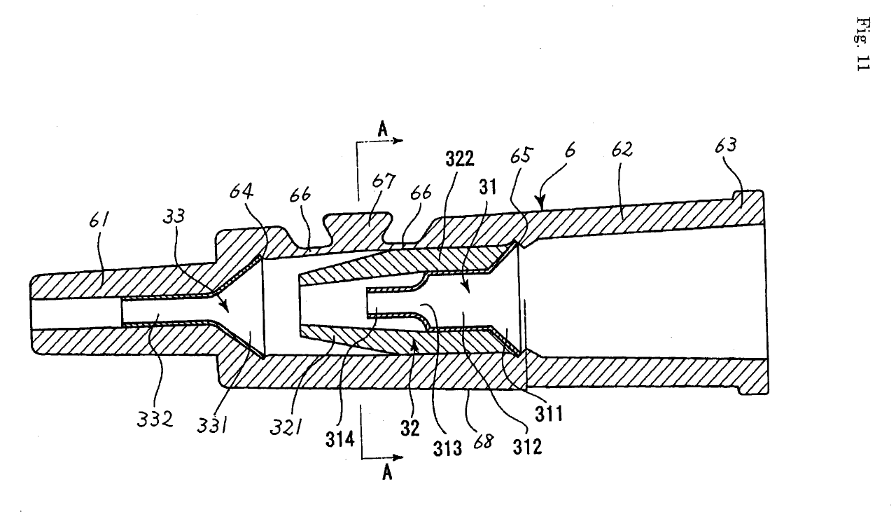

FIG. 11 is a front sectional view of a plug for a catheter according to an embodiment of a medical device of a second aspect of the invention.

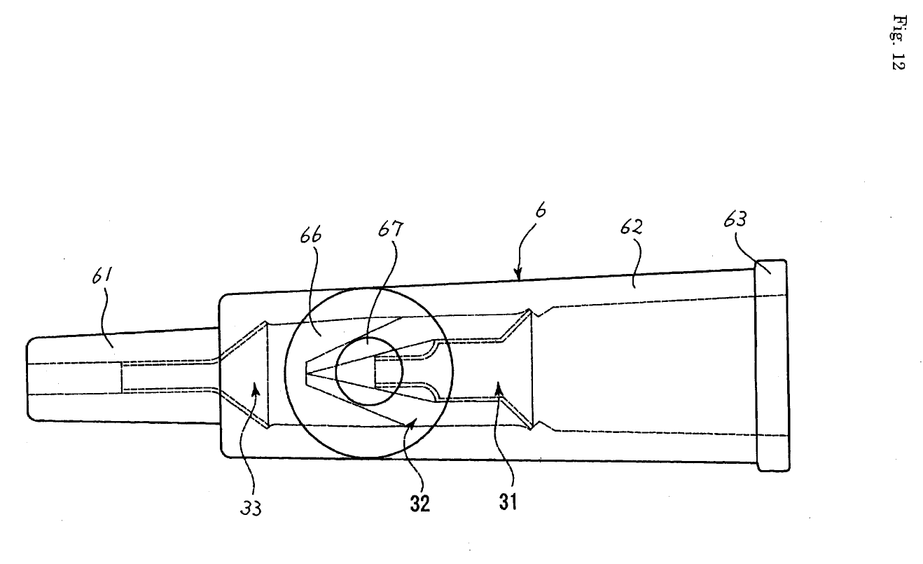

FIG. 12 is a plan view of the plug for a catheter according to the embodiment of the medical device of the second aspect of the invention.

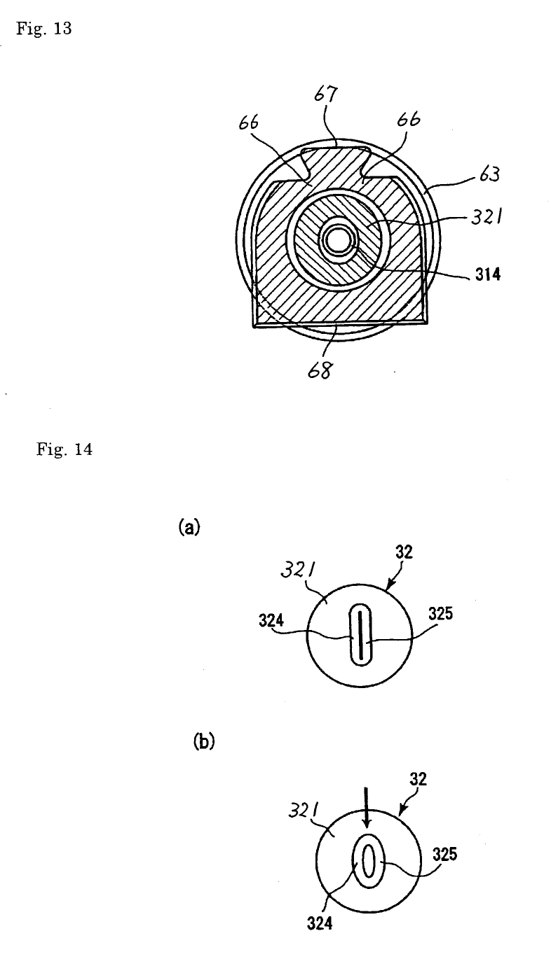

FIG. 13 is a sectional view along the line A-A in FIG. 11.

FIG. 14 illustrates an operation of a check valve included in the plug for a catheter according to the embodiment.

A frame 6 includes a small diameter portion 61, a body 62, and a flange 63, and is made of transparent or translucent soft resin. A flange side of the body 62 is cylindrical, and a small diameter portion side of the body 62 is flat at a bottom 68 and has a thin portion 66 and a button 67 at a top. When a top surface of the button 67 is pressed with a finger, the bottom 68 stably contacts a skin of a patient's arm or thigh, and the thin portion 66 is dented and moves downwards with the frame 6 being not easily moved. Thus, a bottom surface of the thin portion 66 contacts the valve body 32 of the check valve 3 to be pressed downwards.

Preferably, the valve body is made of silicone rubber, urethane rubber, or plastic soft materials, and when a force is applied to the valve body to be elastically deformed, and then the force is removed by releasing the finger, the valve body returns to its original shape. A preferable elastic material has repeatability that allows returning to the original shape even after repeated application and removal of a force. In this embodiment, the valve body 32 is in a “ruling pen” shape, and long sides of two flat plates 324, 325 at a tip are placed perpendicularly to the bottom 68 of the frame 6. Usually, the long sides of the flat plates 324, 325 tightly contact each other. However, when the button 67 is pressed with a finger, the thin portion 66 is flexed to press the valve body 32 downwards, and the flat plates 324, 325 are flexed to be arcuate to leave a clearance between the flat plates 324, 325. Therefore, a fluid passes through the check valve 3. On the other hand, when pressing the button 67 is stopped, the valve body 32 returns to its original shape, the flat plates 324, 325 tightly contact each other, and the fluid cannot pass therebetween. Thus, even if a medical practitioner presses the frame 6 with his/her finger while holding with one hand, the frame 6 does not rotate and move, thereby allowing a stable and smooth operation.

Thirteenth Embodiment (Second Aspect of the Invention)

In unshown another embodiment, a valve body is in a sack shape, and a tip thereof has a cross-shaped slit. When a pressure acting on an inner surface of the valve body is not larger than a pressure acting on an outer surface, a check valve is usually closed. However, when a lateral force is applied to the valve body, the valve body is elastically deformed to leave a clearance at the slit to open the valve body. On the other hand, when application of the lateral force is stopped, the valve body returns to its original shape and is closed.

The embodiments of the plug for a catheter have been described, but the same applies to an indwelling needle for a catheter, an inner needle for a catheter, a wing needle, a plug for a wing needle, an infusion tube, and a plug for an infusion tube.

According to the plugs for a catheter of the twelfth and the thirteenth embodiments of the second aspect of the invention, backflow of blood or a body fluid can be basically prevented, and further, pressing part of a frame with a finger while holding with one hand allows backflow only during pressing the part. Thus, a single medical practitioner can remove an air pool behind a check valve, connect the plug for a catheter to an infusion tube, and create a circuit without mixed air relatively easily.

According to the medical device for humans and animals of the invention, backflow of blood or a body fluid can be basically prevented, and further, pressing part of a frame with a finger while holding with one hand allows backflow only during pressing the part. Thus, a single medical practitioner can collect blood relatively easily without inserting a new injection needle into a patient's skin. This reduces the risk of developing blood infection diseases (such as HIV or hepatitis) caused by the medical practitioner accidentally sticking the needle to his/her finger.

Next, embodiments of a third aspect of the invention will be described.

Fourteenth Embodiment (Third Aspect of the Invention)

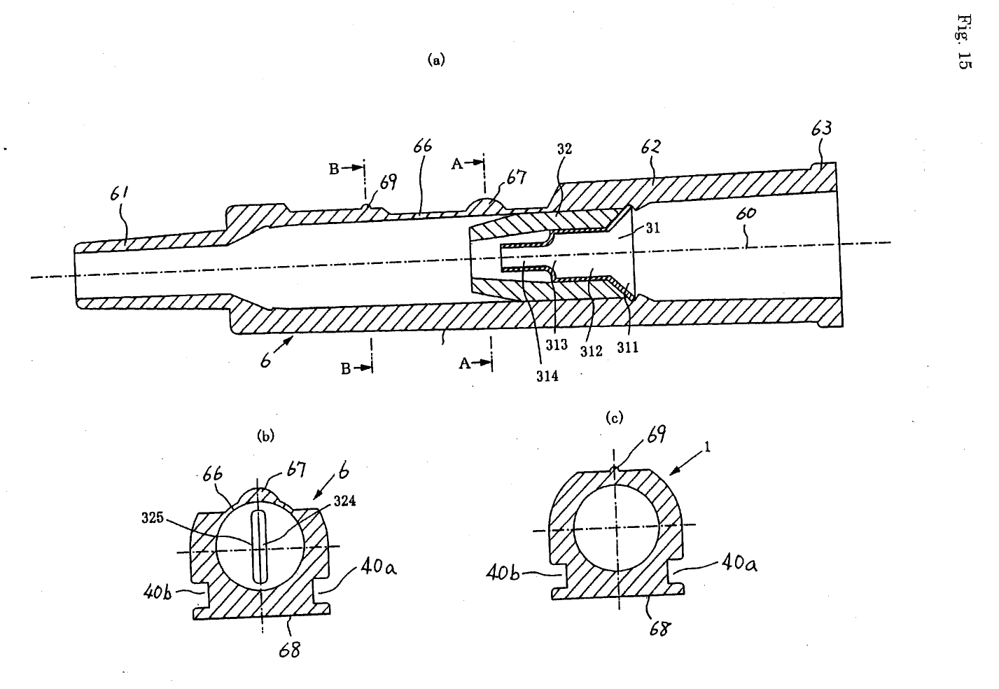

FIG. 15 illustrates a plug for a catheter according to an embodiment of a third aspect of the invention, and (a) is a front sectional view, (b) is a sectional view along the line A-A in (a), and (c) is a sectional view along the line B-B in (a).

A frame 6 of a plug for a catheter includes a small diameter portion 61, a body 62, and a flange 63, and is made of transparent or translucent soft resin. A flange side of the body 62 is cylindrical, and a small diameter portion side of the body 62 is flat at a bottom 68 and has a thin portion 66, a button 67, and a small protrusion 69 at a top. Straight grooves 40a, 40b extending in parallel with a central axis 60 are formed on both sides of the frame 6 near the bottom 68.

A check valve 3 is provided in the frame 6. The check valve 3 includes a check valve holder 31 made of metal, and a valve body 32 made of silicone rubber, natural rubber, or synthetic rubber. The check valve holder 31 includes a hollow conical tapered pipe portion 311, a straight pipe 312 connecting to a small diameter opening of the tapered pipe portion, a rounded portion 313, and a straight pipe 314 connecting to a small diameter opening of the rounded portion. The valve body 32 includes a hollow straight pipe 322 and a “ruling pen” portion 321 connecting thereto. Like a “ruling pen” for drawing, two flat plates 324, 325 are placed with their long sides contacting each other near a tip of the “ruling pen” portion 321.

When a force acting on an outer surface of the valve body 32 (a fluid pressure) is equal to or larger than a force acting on an inner surface (a fluid pressure), the two flat plates 324, 325 tightly contact each other and do not pass any fluid. On the other hand, when the force acting on the inner surface of the valve body 32 (the fluid pressure) is larger than the force acting on the outer surface (the fluid pressure) by a predetermined value or more, the two flat plates 324, 325 are separated to leave a clearance therebetween and open, and a fluid passes through the opening. When a compressive force acts along the long sides of the flat plates 324, 325, the two flat plates 324, 325 are separated to leave a clearance therebetween and open, and a fluid passes through the opening.

The flat plates 324, 325 near the tip of the valve body 32 of the check valve 3 are placed beneath the button 67 with the long sides running vertically.

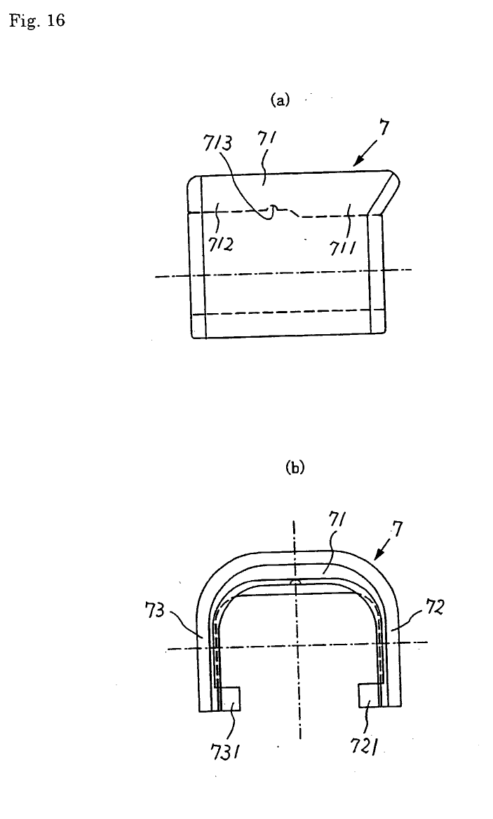

FIG. 16 illustrates a pressing tool of a plug for a catheter according to an embodiment of the third aspect of the invention, and (a) is a front view thereof and (b) is a side view thereof.

A pressing tool 7 is an embodiment of the third aspect of the invention according to claim 4. The pressing tool 7 includes a right side plate 72 and a left side plate 73 extending downwards from both ends of a central plate 71 toward the same side, and an end of the right side plate 72 has a protrusion 721 protruding inwards, and an end of a left side plate 73 also has a protrusion 731 protruding inwards. A bottom surface of the central plate 71 of the pressing tool 7 includes a thick portion 711 and a thin portion 712, and part of the thin portion 712 has an engagement recess 713. Preferably, the central plate 71 has an uneven top surface in order to prevent slide when moving the pressing tool 7 while pressing with a finger. Preferably, rounded corners are provided in order to prevent injury or cut from corners.

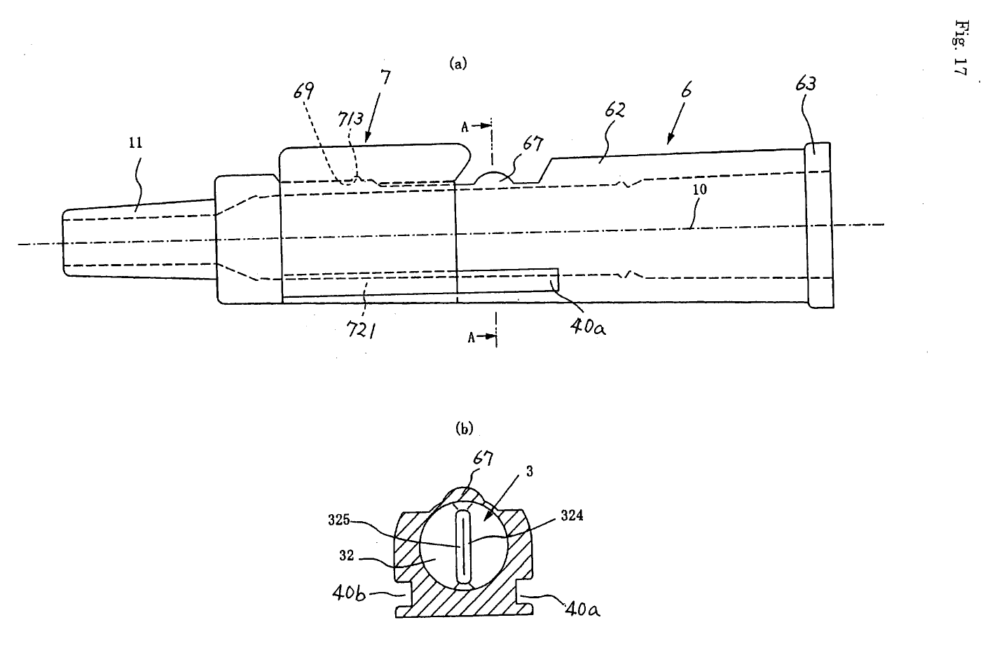

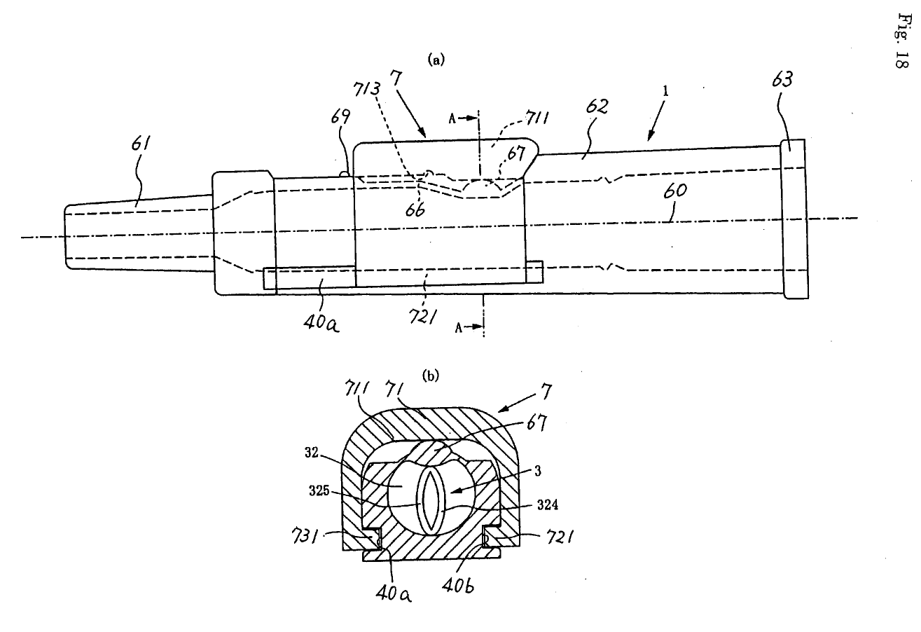

FIGS. 17 and 18 illustrate operations of the plug for a catheter according to the third aspect of the invention.

The pressing tool 7 is placed over the body 62 of the frame 6, the protrusions 721, 731 of the pressing tool 7 are engaged in the straight grooves 40a, 40b of the body 62, and the protrusions 721, 731 are guided and linearly moved along the straight grooves 40a, 40b, then the pressing tool 7 moves over the thin portion 66 along the central axis 60 of the frame 6.

As shown in FIG. 17, when the pressing tool 7 is brought near the small diameter portion 61 of the frame 6, the small protrusion 69 of the frame 6 engages the engagement recess 713 of the pressing tool 7. In a normal state, the pressing tool 7 is in this position, and when a fluid pressure acting on an inner surface of the check valve is not larger than a fluid pressure acting on an outer surface by a predetermined value or more, as shown in FIG. 17(b), the long sides of the flat plates 324, 325 near the tip of the valve body tightly contact each other with respect to the section along the line A-A in (a).

On the other hand, as shown in FIG. 18, when the pressing tool 7 is brought near the flange 63, the small protrusion 69 is first disengaged from the engagement recess 713, then a bottom surface of the thick portion 711 of the central plate 71 of the pressing tool 7 abuts the button 67 of the frame 6 to press the button 67 downwards. Thus, the thin portion 66 of the body 62 of the frame 6 is also flexed downwards, and a compressive force keeps acting along the long sides of the flat plates 324, 325 near the tip of the valve body 32 of the check valve 3. Then, the two flat plates 324, 325 are separated to leave a clearance therebetween and open. This allows a fluid to freely pass through the opening. Thus, there is no need for a medical practitioner such as a doctor or a nursing person to always stay near the plug for a catheter, to continue pressing with his/her finger, or to replace with a plug with no check valve. Further, there is no need for the patient to continue pressing with his/her finger.

Fifteenth Embodiment (Third Aspect of the Invention)

In another embodiment, a pressing tool includes a hollow cylinder that is threaded on its side surface, and a threaded cap that thread into the hollow cylinder. A button of a plug for a catheter is placed in the hollow cylinder, and the side surface of the hollow cylinder extends in parallel with a protruding direction of the button. A bottom surface of the threaded cap can press a top of the button.

When the threaded cap is rotated in a forward direction, the threaded cap moves downwards in a spiral along the hollow cylinder to press the button of the plug for a catheter. Then, the valve body of the check valve placed in the plug for a catheter and beneath the button is kept pressed near the tip.

On the other hand, when the threaded cap is rotated in a reverse direction, the threaded cap moves upwards in a reverse spiral along the hollow cylinder, and the button of the plug for a catheter is not pressed and returns to its original position.

In order to prevent loss of the threaded cap, it is preferable to connect the threaded cap and the frame of the plug for a catheter by a flexible member such as a cord, a string, a tube, a line, or a tape.

In order to facilitate pressing the button of the plug for a catheter with a finger, it is preferable to notch part of the side surface of the hollow cylinder or provide a low side surface to the hollow cylinder.

The threaded cap may be able to be rotated half or a quarter of a turn in the forward and reverse directions, and have a structure in which the threaded cap is not detached and separated from the hollow cylinder as a rule. Thus, even if the threaded cap or the frame of the plug for a catheter are accidentally pressed resulting from rolling over of a patient, the button is not pressed. Further, open/close switching of the check valve is facilitated. It is preferable to mark part of a circumference or part of an outer side surface of the threaded cap so as to facilitate distinguishing between an open state and a closed state.

Sixteenth Embodiment (Third Aspect of the Invention)

In a further embodiment, a pressing tool includes an elastic belt and engaging tools. One end of the elastic belt is fastened near a button of a plug for a catheter. One of the engaging tools is mounted to the other end of the elastic belt. The other of the engaging tools is mounted on an extending line on a button side of a line connecting one end of the elastic belt and the button.

When one of the engaging tools and the other of the engaging tool are engaged with a portion around a center of the elastic belt in contact with a top surface of the button, the portion around the center of the elastic belt presses the button. Then, the valve body of the check valve placed in the plug for a catheter and beneath the button is kept pressed near the tip.

The embodiments of the plug for a catheter according to the invention have been illustrated and described, but the same applies to other medical devices for humans or animals.

In the medical device according to the third aspect of the invention, the check valve is provided in the frame, thus, as a rule, a fluid flows in a single direction. Further, the check valve is positioned in the flexible portion of the frame, thus the check valve opens when the flexible portion is pressed to allow the fluid to flow in both directions.

Further, the medical device for humans or animals according to the invention has the pressing tool for continuously pressing the flexible portion of the frame, thus the check valve can be kept open even if the medical practitioner does not stay near the patient, or the patient does not continue pressing the flexible portion with his/her finger. This reduces burdens on the medical practitioner or the patient, and reduces the number of medical accidents since a doze during pressing with the finger causes no stop of pressing the flexible portion.

The medical device according to the third aspect of the invention allows repeated switching between the open state and a normal check valve state by operating a pressing portion, and is thus cost-effective compared to throwaway or replacement.

Now, embodiments of a fourth aspect of the invention will be described.

Seventeenth Embodiment (Fourth Aspect of the Invention)

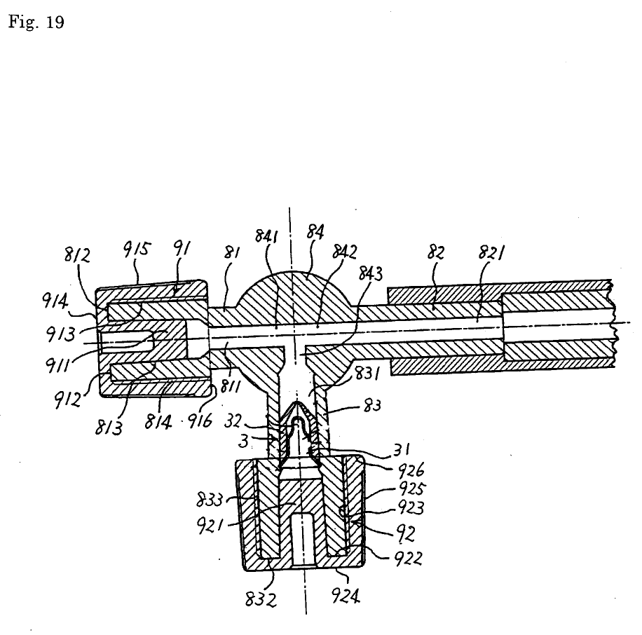

FIG. 19 is a plan view of a side branch pipe according to a seventeenth embodiment.

A side branch pipe includes a first straight pipe 81, a second straight pipe 82, a side branch 83, and a confluence 84. The first straight pipe 81 and the second straight pipe 82 are arranged on a line. The first straight pipe 81 and the side branch 83 intersect at the confluence 84. The second straight pipe 82 and the side branch 83 also intersect at the confluence 84. An inner pipe 811 connects to an inner pipe 841, an inner pipe 821 connects to an inner pipe 842, an inner pipe 831 connects to an inner pipe 843, and the inner pipes 841, 842, 843 are in communication.

A straight pipe cap 91 threads into a free end 812 of the first straight pipe 81 fluid-tightly. The straight pipe cap 91 includes an inner bottom surface 912, an outer bottom surface 914, an inner peripheral surface 913, an annular end 916 of an outer peripheral surface 915, and a fitting protrusion 911. The inner peripheral surface 913 is threaded. An outer peripheral surface 814 of the first straight pipe 81 is correspondingly threaded. When the straight pipe cap 91 starts threading into the free end 812 of the first straight pipe 81, the fitting protrusion 911 is inserted into the first straight pipe 81, the inner peripheral surface 813 of the first straight pipe 81 starts tightly contacting the outer peripheral surface of the fitting protrusion 911, and when the straight pipe cap 91 is further threaded into fluid-tightly, the free end 812 of the first straight pipe 81 and the inner bottom surface of the straight pipe cap 91 tightly contact each other.

A side branch cap 92 threads into a free end 832 of the side branch 83 fluid-tightly. The side branch cap 92 includes an inner bottom surface 922, an outer bottom surface 924, an inner peripheral surface 923, an outer peripheral surface 925, an annular end 926, and a fitting protrusion 921. The inner peripheral surface 923 is threaded. An outer peripheral surface 833 of the side branch 83 is correspondingly threaded. When the side branch cap 92 starts threading into the free end 832 of the side branch 83, the fitting protrusion 921 is inserted into the side branch 83, the outer peripheral surface 833 of the side branch 83 starts tightly contacting the outer peripheral surface of the fitting protrusion 921, and when the side branch cap 92 is further threaded into fluid-tightly, the free end 832 of the side branch 83 and the inner bottom surface 922 of the side branch cap 92 tightly contact each other.

An outer diameter of the second straight pipe is substantially constant, and the second straight pipe can be connected to an unshown tube by being fitted to an end of the tube or using an adhesive.

Preferably, the first straight pipe, the second straight pipe, the side branch, and the confluence are made of transparent resin. Transparent materials allow blood or a drug solution flowing in the inner pipes to be visible.

According to the seventeenth embodiment, after detaching the side branch cap 92, simply connecting a tube connected to a drug solution bag to the free end of the side branch causes the drug solution to be supplied to the first straight pipe 81, and disconnecting the tube stops the supply of the solution from the side branch.

Eighteenth Embodiment (Fourth Aspect of the Invention)

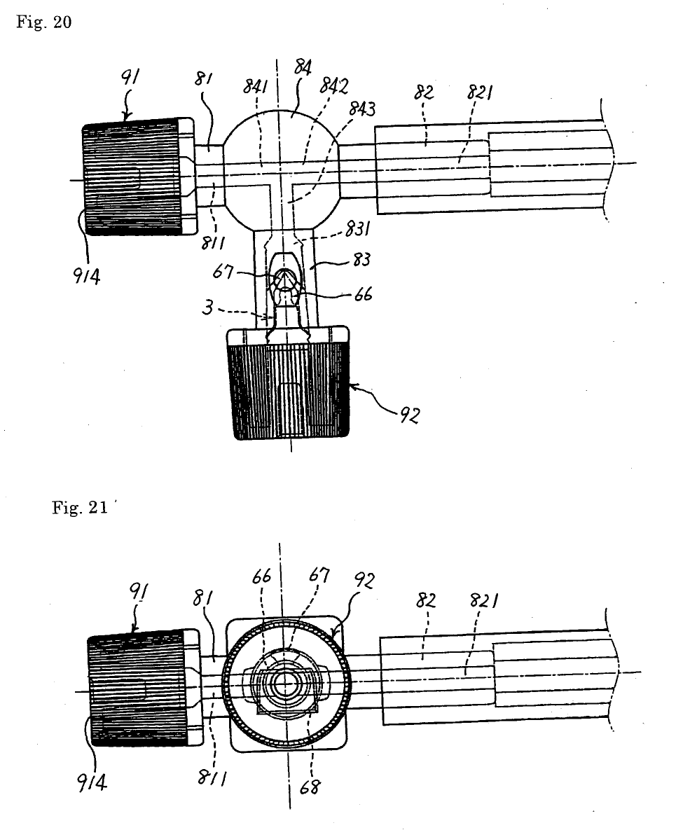

FIG. 20 is a plan view of a side branch pipe according to an eighteenth embodiment. FIG. 21 is a front view of the side branch pipe according to the eighteenth embodiment.

Unlike the side branch pipe of the seventeenth embodiment, in the side branch pipe of the eighteenth embodiment, a side branch 83 has a thin portion 66 at a top of a side surface near a check valve 3, has a button 67, and has a flat portion 68 at a bottom. When a top surface of the button 67 is pressed with a finger with the flat portion 68 in tight contact with a patient's skin, the thin portion 66 is elastically dented and moves downwards. Thus, a bottom surface of the thin portion 66 contacts the check valve 3, and a lateral force is applied to the check valve. Therefore, the valve body is deformed to open a slit and to allow backflow.

Preferably, the valve body is made of silicone rubber, urethane rubber, or plastic soft materials, and when a force is applied to the valve body to be elastically deformed, and then the force is removed by releasing the finger, the valve body returns to its original shape. A preferable elastic material has repeatability that allows returning to the original shape even after repeated application and removal of a force. In the eighteenth embodiment, the valve body is in a “ruling pen” shape, and long sides of two flat plates at a tip are placed perpendicularly to the bottom of the side branch pipe. Usually, the long sides of the two flat plates tightly contact each other. However, when the button 67 is pressed with a finger, the thin portion 66 is flexed to press the valve body downwards, and the flat plates are flexed to be arcuate to leave a clearance between the two flat plates. Therefore, a fluid passes through the check valve 3. On the other hand, when pressing the button 67 is stopped, the valve body returns to its original shape, the two flat plates tightly contact each other, and the fluid cannot pass therebetween. Thus, even if a medical practitioner presses the side branch pipe with his/her finger while holding with one hand, the side branch pipe does not rotate and move, thereby allowing a stable and smooth operation.

In unshown another embodiment, a valve body is in a sack shape, and a tip thereof has a cross-shaped slit. When a pressure acting on an inner surface of the valve body is not larger than a pressure acting on an outer surface, a check valve is usually closed. However, when a lateral force is applied to the valve body, the valve body is elastically deformed to leave a clearance at the slit to open the valve body. On the other hand, when application of the lateral force is stopped, the valve body returns to its original shape and is closed.

Nineteenth Embodiment (Fourth Aspect of the Invention)

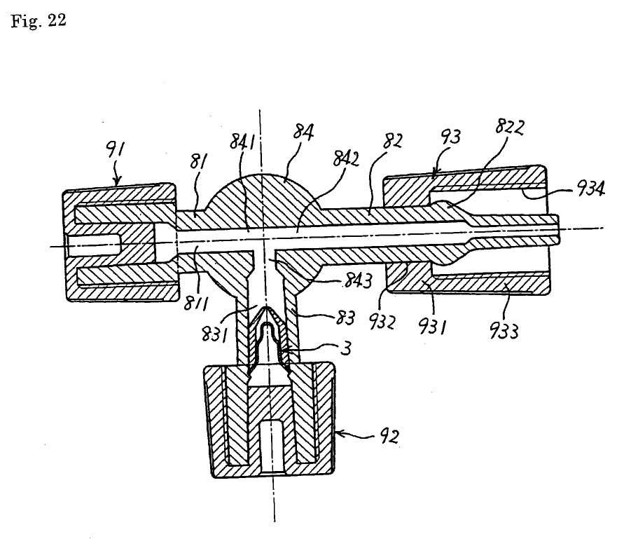

FIG. 22 is a plan view of a side branch pipe according to a nineteenth embodiment.

The nineteenth embodiment is substantially the same as the seventeenth embodiment except for a cap nut 93 being mounted to a second straight pipe 82 rotatably and somewhat movably along an axis. The cap nut 93 includes an annular bottom 931 having an inner diameter substantially identical to an outer diameter of the second straight pipe 82, and a side peripheral portion 933 vertically extending from an outer periphery of the annular bottom 931. An inner peripheral surface of the side peripheral portion 933 is threaded. Further, unlike the seventeenth embodiment, the second straight pipe 82 has a large diameter protrusion 822 at its center. An outer diameter of the large diameter protrusion 822 is larger than an inner diameter of the annular bottom 931 of the cap nut 93, thus the cap nut 93 cannot move beyond the large diameter protrusion 822, but between a confluence 84 and the large diameter protrusion 822, an inner peripheral surface 932 of the annular bottom 931 can rotate and linearly move in contact with an outer peripheral surface of the second straight pipe 82.

Twentieth Embodiment (Fourth Aspect of the Invention)

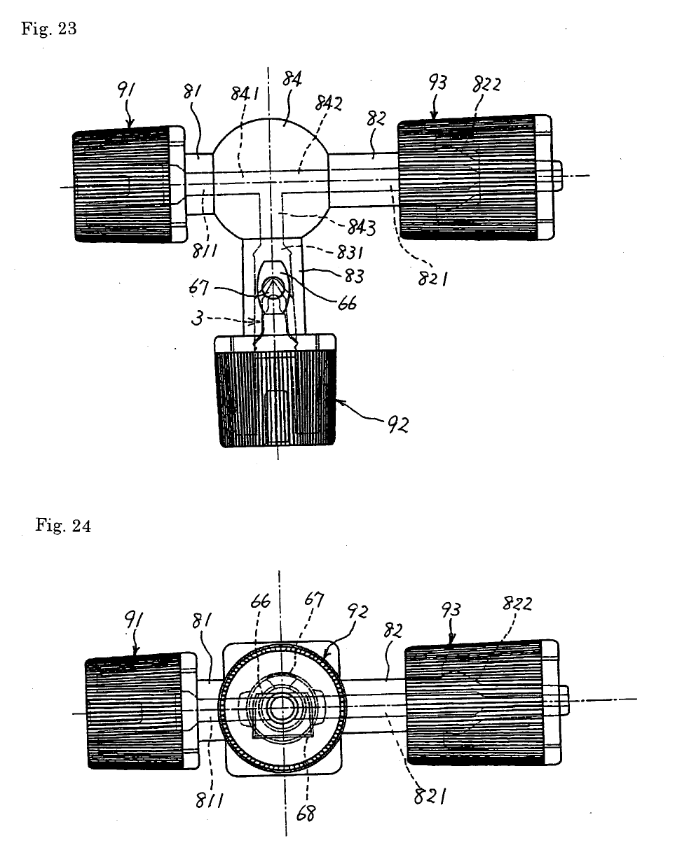

FIG. 23 is a plan view of a side branch pipe according to a twentieth embodiment, and FIG. 24 is a front view of the side branch pipe according to the twentieth embodiment.

Like the eighteenth embodiment, in the twentieth embodiment, a side branch 83 has a thin portion 66 at a top of a side surface near a check valve 3, has a button 67, and has a flat portion 68 at a bottom. Further, like the nineteenth embodiment, in the twentieth embodiment, a second straight pipe 82 has a large diameter protrusion 822 at its center, and a cap nut 93 is mounted to the second straight pipe 82.

The side branch pipe of the nineteenth embodiment and the side branch pipe of the nineteenth embodiment can be joined such that the second straight pipe of the former and the first straight pipe of the latter are arranged on a line to cause the cap nut of the former to thread into the outer peripheral surface of the latter. Likewise, the side branch pipe of the twentieth embodiment and the side branch pipe of the twentieth embodiment can be joined, and the side branch pipe of the nineteenth embodiment and the side branch pipe of the twentieth embodiment can be joined.

Twenty-First Embodiment (Fourth Aspect of the Invention)

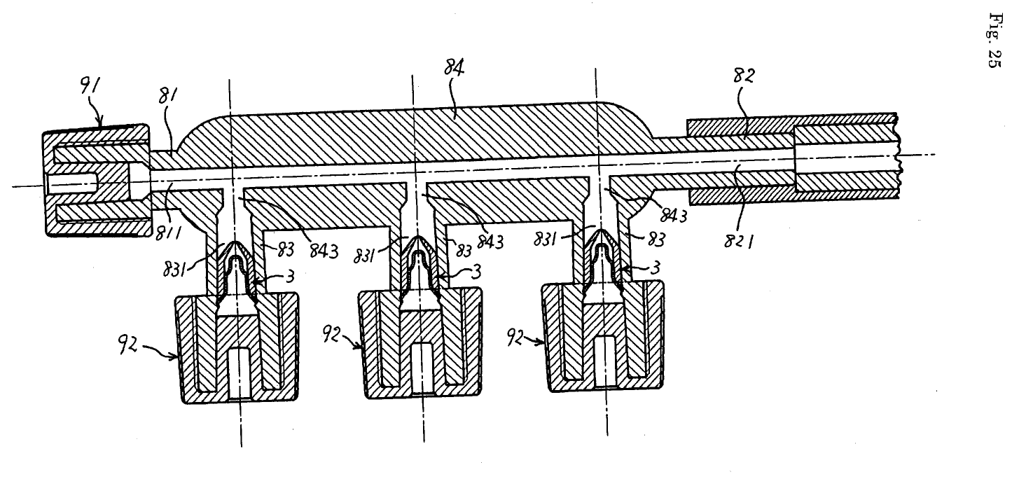

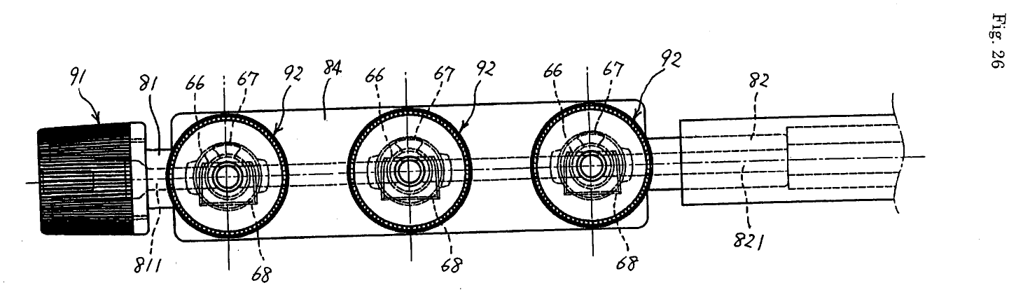

FIG. 25 is a plan view of a side branch pipe according to a twenty-first embodiment, and FIG. 26 is a front view of the side branch pipe according to a twenty-first embodiment.

A first straight pipe 81 and a second straight pipe 82 are arranged on a line, a confluence 84 extends along the line and has three side branches 83 intersecting the line. The three side branches are parallel to each other and placed in the same direction with respect to the line in one plane.

Each side branch 83 has a thin portion and a button at part of a side surface near a check valve like the side branch pipe of the eighteenth embodiment or the side branch of the side branch pipe of the twentieth embodiment, but the side branch 83 may have a structure simply including a check valve like the side branch pipe of the seventeenth embodiment or the side branch of the side branch pipe of the nineteenth embodiment.

For easy identification of each side branch 83, part of the side branch, a side branch cap, or both may be colored for color-coding.

The side branch pipe of the twentieth embodiment allows drug solutions from various drug solution bags to be mixed to flow at the same time, facilitates switching, and prevents switching errors since each side branch can be quickly identified.

Modification of the twenty-first embodiment (fourth aspect of the invention)

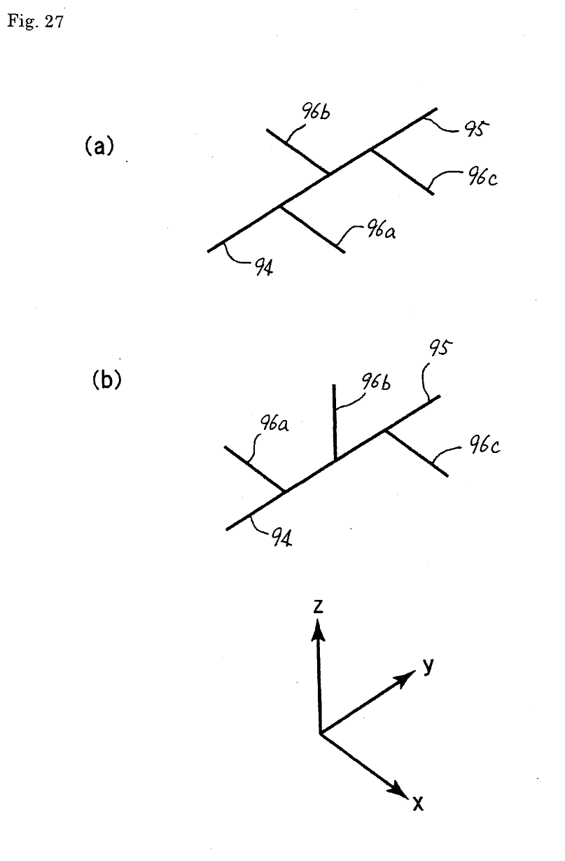

FIG. 27 is a schematic view of a modification of the twenty-first embodiment. In (a), three side branches 96 are in one plane, side branches 96a, 96c extend in the same direction with respect to a line connecting a first straight pipe 94 and a second straight pipe 95, while a side branch 96b extends in an opposite direction. Jam or entanglement of connected tubes can be prevented. In (b), side branches 96a, 96c among three side branches 96 are in one plane, but extend in opposite directions with respect to the line. The side branch 96b intersects the line and the side branch 96a, and is perpendicular to the plane. This prevents a mix-up between the side branches 96a, 96b, 96c.

Twenty-Second Embodiment (Fourth Aspect of the Invention)

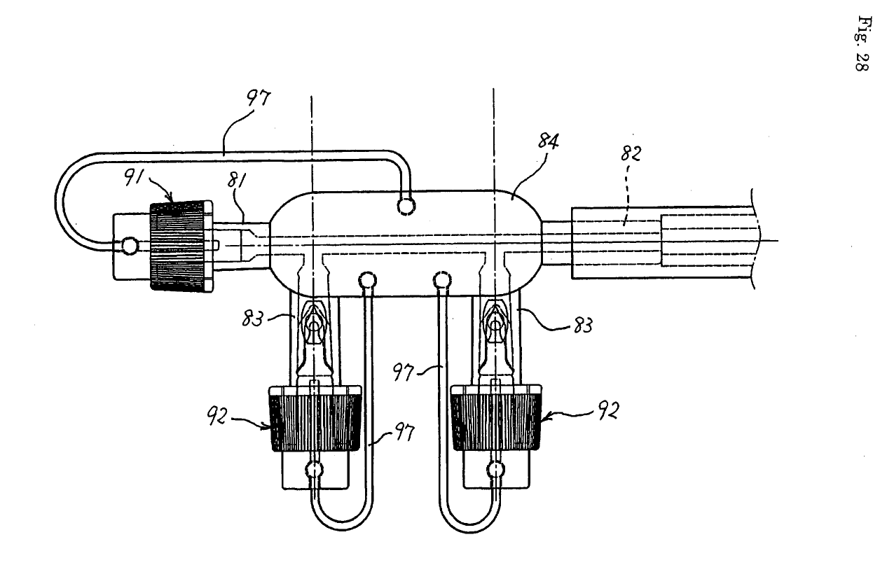

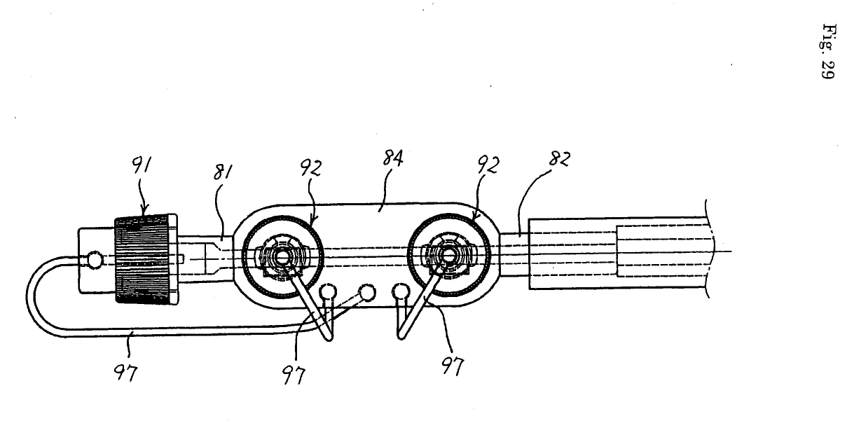

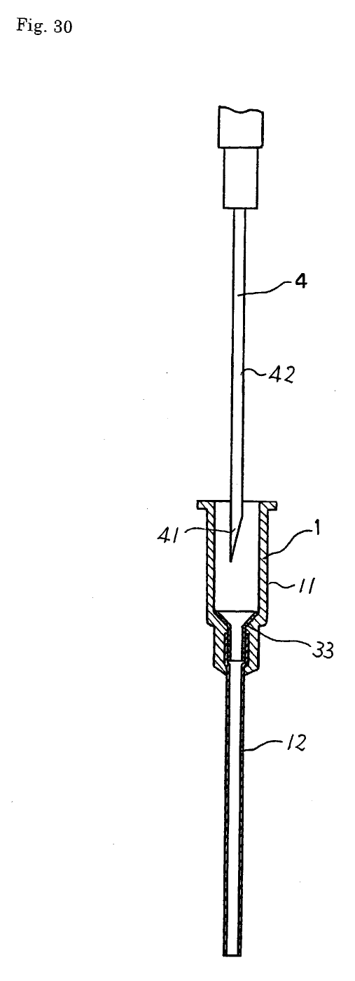

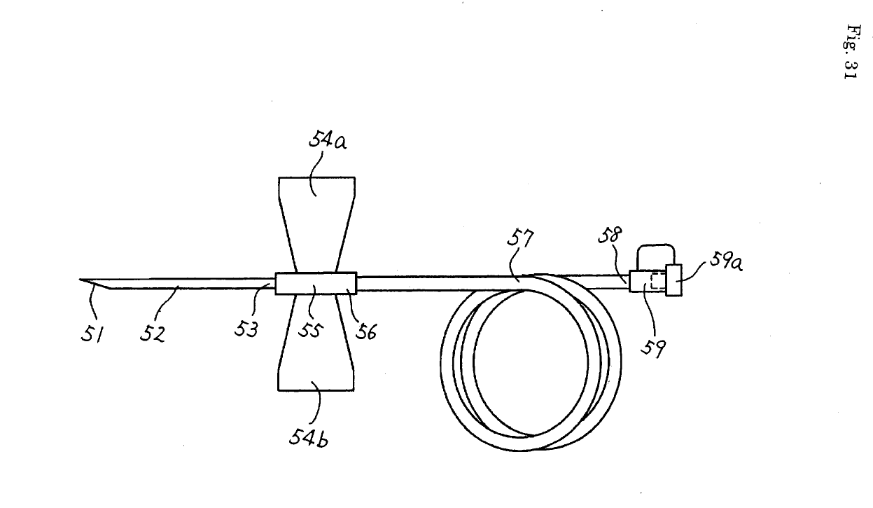

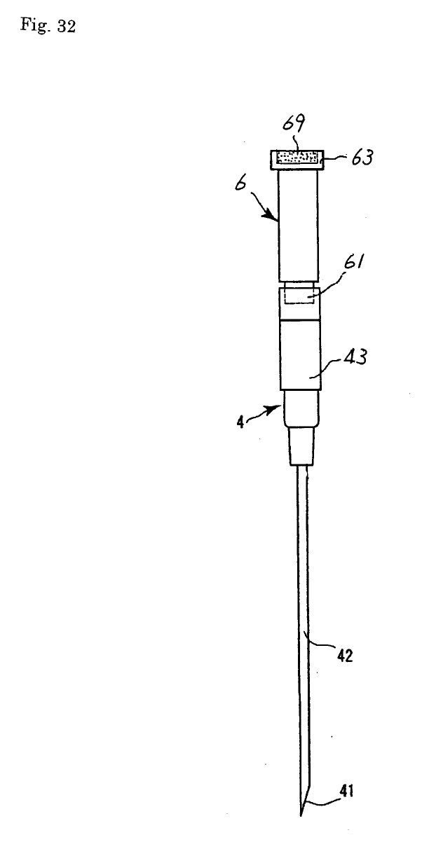

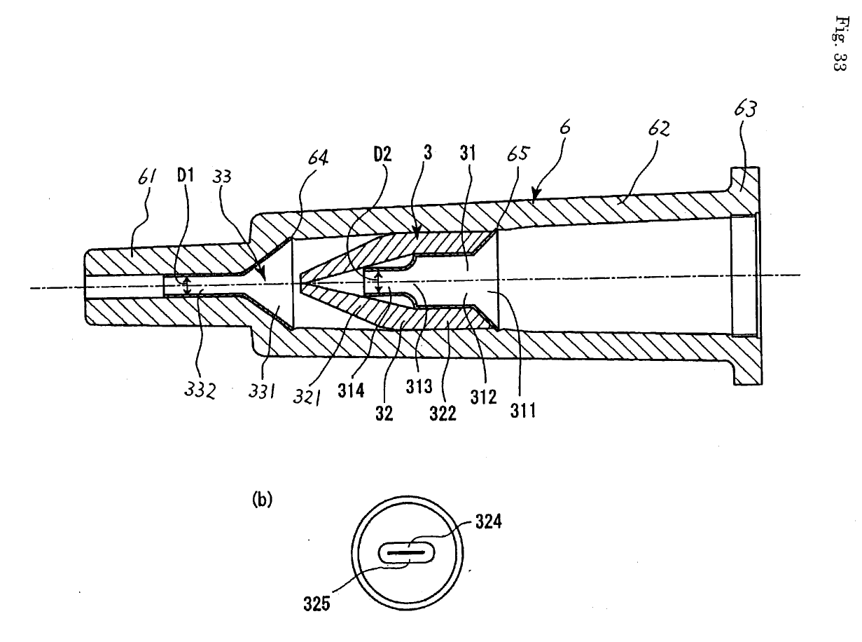

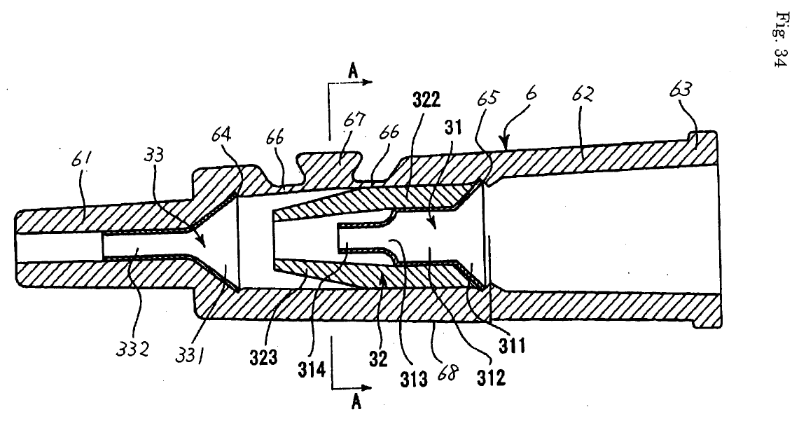

FIG. 28 is a plan view of a side branch pipe according to a twenty-second embodiment, and FIG. 29 is a front view of the side branch pipe according to the twenty-second embodiment.

A straight pipe cap 91 and a side branch cap 92 are connected to a confluence 84 by a flexible wire 97 made of plastic, a fiber string, or metal. Connecting the straight pipe cap 91 and the side branch cap 92 to the confluence 84 by the wire 97 solves problems of loss of the straight pipe cap 91 and the side branch cap 92, or of taking time or trouble in finding them. The straight pipe cap 91 and the side branch cap 92 may be differently colored, or may be colored as the same as a straight pipe cap that threads into part of a corresponding straight pipe or a side branch cap that threads into part of a corresponding side branch.

According to the medical side branch pipe of the fourth invention, simply connecting a tube to the side branch allows a fluid to flow from the tube to the confluence without backflow, and detaching the tube stops the flow of the fluid, thus current situations of connection or a flowing fluid can be quickly perceived, preventing operating errors to reduce the number of medical accidents.

According to the medical side branch pipe of the fourth invention, a plurality of side branches protrude from the confluence, simply connecting a tube to each side branch allows a fluid to flow from the tube to the confluence without backflow, and detaching the tube stops the flow of the fluid, thus even if a plurality of drug solutions are administered to a patient at the same time or by switching, current situations of connection or a flowing fluid can be quickly perceived, preventing operating errors to reduce the number of medical accidents.

Finally, embodiments of a fifth aspect of the invention will be described.

Twenty-Third Embodiment (Fifth Aspect of the Invention)

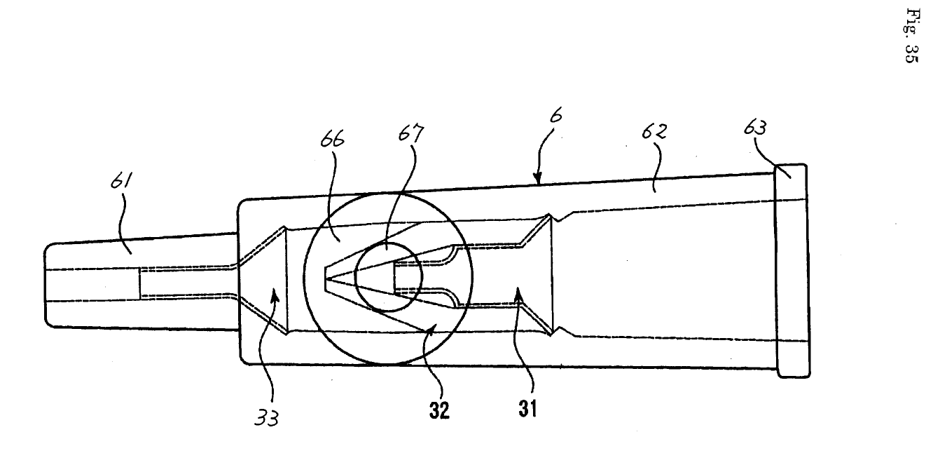

FIG. 35 is a plan view of a plug for a catheter according to a twenty-third embodiment of the invention.

A frame 6 includes a small diameter portion 61, a body 62, and a flange 63, and is made of transparent or translucent soft resin. A flange side of the body 62 is cylindrical, and a small diameter portion side of the body 62 is flat at a bottom and has a thin portion 66 and a button 67 at a top. When a top surface of the button 67 is pressed with a finger, the bottom stably contacts a skin of a patient's arm or thigh, and the thin portion 66 is dented and moves downwards with the frame 6 being not easily moved. Thus, a bottom surface of the thin portion 66 contacts the valve body 32 of the check valve 3 to be pressed downwards.

The button 67 is coated with fluorescent paint 98 in a circular shape, which looks yellow in the light to the naked eye, and glows in the dark with the fluorescence. Using fluorescent paints of various colors (red, orange, green, yellow, blue, or the like) allows the plug for a catheter to be easily identified in the dark.

As another embodiment, the button 67 may be coated with the fluorescent paint in the shape of a target with concentric circles, a star, a heart, a petal, a square or a triangle.

Preferably, the valve body is made of silicone rubber, urethane rubber, or plastic soft materials, and when a force is applied to the valve body to be elastically deformed, and then the force is removed by releasing the finger, the valve returns to its original shape. A preferable elastic material has repeatability that allows returning to the original shape even after repeated application and removal of a force. In this embodiment, the valve body 32 is in a “ruling pen” shape, and long sides of two flat plates at a tip are placed perpendicularly to the bottom of the frame 6. Usually, the long sides of the flat plates tightly contact each other. However, when the button 67 is pressed with a finger, the thin portion 66 is flexed to press the valve body 32 downwards, and the flat plates are flexed to be arcuate to leave a clearance between the flat plates. Therefore, a fluid passes through the check valve 3. On the other hand, when pressing the button 67 is stopped, the valve body 32 returns to its original shape, the flat plates tightly contact each other, and the fluid cannot pass therebetween. Thus, even if a medical practitioner presses the frame 6 with his/her finger while holding with one hand, the frame 6 does not rotate and move, thereby allowing a stable and smooth operation.

Twenty-Fourth Embodiment (Fifth Aspect of the Invention)

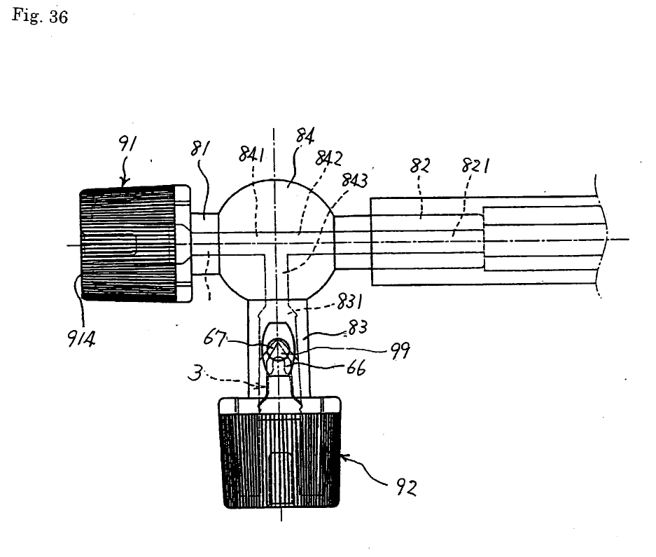

FIG. 36 is a plan view of a medical side branch pipe according to a twenty-fourth embodiment of a fifth aspect of the invention.

A side branch pipe includes a first straight pipe 81, a second straight pipe 82, a side branch 83, and a confluence 84. The first straight pipe 81 and the second straight pipe 82 are arranged on a line. The first straight pipe 81 and the side branch 83 intersect at the confluence 84. The second straight pipe 82 and the side branch 83 also intersect at the confluence 84. An inner pipe 811 connects to an inner pipe 841, an inner pipe 821 connects to an inner pipe 842, an inner pipe 831 connects to an inner pipe 843, and the inner pipes 841, 842, 843 are in communication.

A straight pipe cap 91 threads into a free end of the first straight pipe 81 fluid-tightly. The straight pipe cap 91 includes an inner bottom surface, an outer bottom surface 914, an inner peripheral surface, an outer peripheral surface, an annular end, and a fitting protrusion. The inner peripheral surface is threaded. An outer peripheral surface of the first straight pipe 81 is correspondingly threaded. When the straight pipe cap 91 starts threading into the free end of the first straight pipe 81, the fitting protrusion is inserted into the first straight pipe 81, the inner peripheral surface of the first straight pipe 81 starts tightly contacting the outer peripheral surface of the fitting protrusion, and when the straight pipe cap 91 is further threaded into fluid-tightly, the free end of the first straight pipe 81 and the inner bottom surface of the straight pipe cap 91 tightly contact each other.

A side branch cap 92 threads into a free end of the side branch 83 fluid-tightly. The side branch cap 92 includes an inner bottom surface, an outer bottom surface, an inner peripheral surface, an outer peripheral surface, an annular end, and a fitting protrusion. The inner peripheral surface is threaded. An outer peripheral surface of the side branch 83 is correspondingly threaded. When the side branch cap 92 starts threading into the free end of the side branch 83, the fitting protrusion is inserted into the side branch 83, the inner peripheral surface of the side branch 83 starts tightly contacting the outer peripheral surface of the fitting protrusion, and when the side branch cap 92 is further threaded into fluid-tightly, the free end of the side branch 83 and the inner bottom surface of the side branch cap 92 tightly contact each other. A check valve 3 is provided in the side branch, and passes a fluid from the free end of the side branch 83 to the confluence 84, but does not pass a fluid in a reverse direction. The side branch 83 has a thin portion 66 at a top of a side surface near the check valve 3, has a button 67, and has a flat portion at a bottom. When a top surface of the button 67 is pressed with a finger with the flat portion in tight contact with a patient's skin, the thin portion 66 is elastically dented and moves downwards. Thus, a bottom surface of the thin portion 66 contacts the check valve 3, and a lateral force is applied to the check valve. Therefore, the valve body 32 is deformed to open the slit and to allow backflow.

Preferably, the valve body is made of silicone rubber, urethane rubber, or plastic soft materials, and when a force is applied to the valve body to be elastically deformed, and then the force is removed by releasing the finger, the valve body returns to its original shape. A preferable elastic material has repeatability that allows returning to the original shape even after repeated application and removal of a force. In this embodiment, the valve body 32 is in a “ruling pen” shape, and long sides of two flat plates at a tip are placed perpendicularly to the bottom of the side branch pipe. Usually, the long sides of the two flat plates tightly contact each other. However, when the button 67 is pressed with a finger, the thin portion 66 is flexed to press the valve body 32 downwards, and the flat plates are flexed to be arcuate to leave a clearance between the two flat plates. Therefore, a fluid passes through the check valve 3. On the other hand, when pressing the button 67 is stopped, the valve body 32 returns to its original shape, the two flat plates tightly contact each other, and the fluid cannot pass therebetween. Thus, even if a medical practitioner presses the side branch pipe with his/her finger while holding with one hand, the side branch pipe does not rotate and move, thereby allowing a stable and smooth operation.

A fluorescent adhesive sheet 99 is affixed to the button 67, a top surface of the fluorescent adhesive sheet 99 being coated with fluorescent paint and a rear surface thereof being coated with an adhesive.

The fluorescent adhesive sheet is in the shape of a target with concentric circles, a circle, a star, a heart, a triangle, a rectangle, or an arrow.

Twenty-Fifth Embodiment (Fifth Aspect of the Invention)

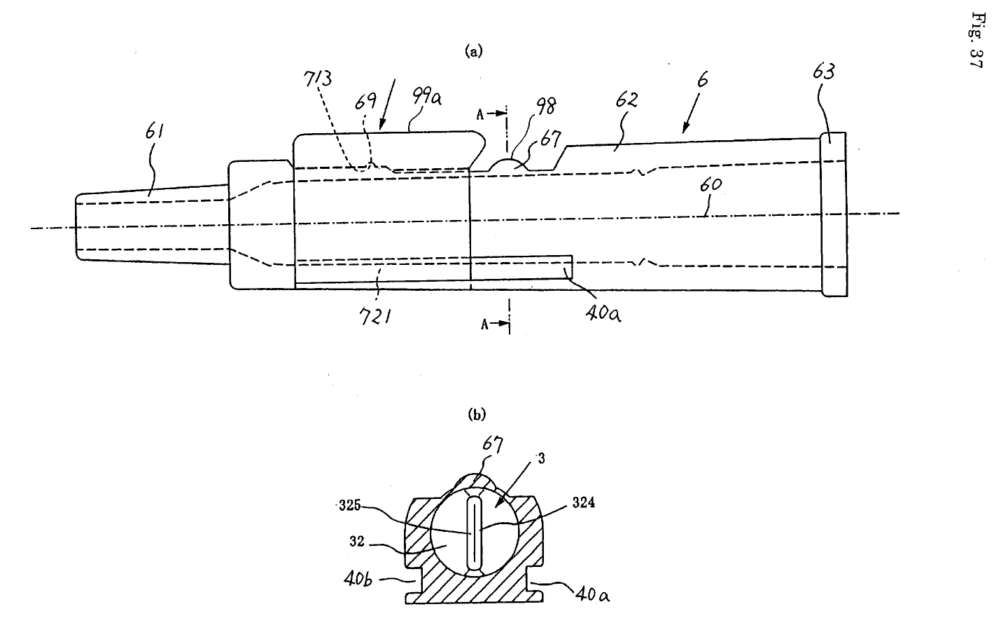

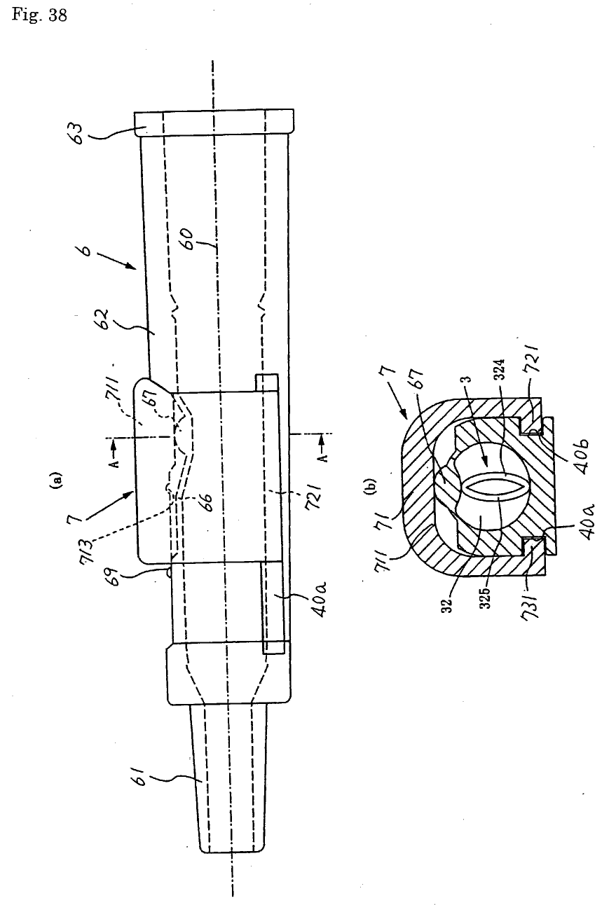

FIGS. 37 and 38 are front views of a plug for a catheter according to a twenty-fifth embodiment of a fifth aspect of the invention.

A pressing tool 7 is placed over a body 62 of a frame 6, protrusions 721, 731 of the pressing tool 7 are engaged in the straight grooves 40a, 40b of the body 62, and the protrusions 721, 731 are guided and linearly moved along the straight grooves 40a, 40b, then the pressing tool 7 moves over the thin portion 66 along the central axis 60 of the frame 6.

As shown in FIG. 37, when the pressing tool 7 is brought near a small diameter portion 61 of the frame 6, a small protrusion 69 of the frame 6 engages an engagement recess 713 of the pressing tool 7. In a normal state, the pressing tool 7 is in this position, and when a fluid pressure acting on an inner surface of a check valve is not larger than a fluid pressure acting on an outer surface by a predetermined value or more, as shown in FIG. 37(b), long sides of flat plates 324, 325 near the tip of a valve body tightly contact each other with respect to the section along the line A-A in (a)

On the other hand, as shown in FIG. 38, when the pressing tool 7 is brought near the flange 63, the small protrusion 69 is first disengaged from the engagement recess 713, then a bottom surface of the thick portion 711 of the central plate 71 of the pressing tool 7 abuts the button 67 of the frame 6 to press the button 67 downwards. Thus, the thin portion 66 of the body 62 of the frame 6 is also flexed downwards, and a compressive force keeps acting along the long sides of the flat plates 324, 325 near the tip of the valve body 32 of the check valve 3. Then, the two flat plates 324, 325 are separated to leave a clearance therebetween and open.

The button 67 is coated with fluorescent paint 98, and a reflector adhesive sheet 99a is affixed to a top surface of a central plate of the pressing tool 7. A top surface of the reflector adhesive sheet is an aluminum reflector, and a rear surface thereof is coated with an adhesive.

When the fluorescence of the button 67 and the reflection light of the pressing tool are both confirmed in a close range, the check valve is in a normal state. When the reflection light of the pressing tool 7 only is confirmed, the pressing tool is placed on the button 67, and the check valve is in an open state.

According to the medical device of the fifth aspect of the invention, even during a light being off, the position to be pressed with the finger (such as the button) can be easily found, and the position or the state of the pressing tool can be easily perceived, thus facilitating the medical practitioner's work during the night and preventing medical accidents.

What is claimed is:

1. A medical device comprising a check valve therein,
the check valve comprising:
a valve holder that has a tapered pipe, and a small diameter pipe connecting to a small diameter opening of the tapered pipe and extending outwards, a large diameter opening of the tapered pipe being fluid-tightly fitted to an inner surface of a side branch; and
a flexible valve body that is fitted to an outer peripheral surface of the small diameter pipe of the valve holder, and has a slit that is opened by a fluid pressure from a large diameter opening side of the tapered pipe, but closed by a fluid pressure from a small diameter pipe side.

2. A medical device comprising:
a flexible portion; and
a check valve in the flexible portion,
the check valve comprising:
a valve holder that has a tapered pipe, and a small diameter pipe connecting to a small diameter opening of the tapered pipe and extending outwards, a large diameter opening of the tapered pipe being fluid-tightly fitted to an inner surface of a side branch; and
a flexible valve body that is fitted to an outer peripheral surface of the small diameter pipe of the valve holder, and has a slit that is opened by a fluid pressure from a large diameter opening side of the tapered pipe, or by a lateral pressure, but closed by a fluid pressure from a small diameter pipe side,
the flexible portion being pressed to press the check valve perpendicularly to a central axis, thus the check valve being deformed and opened.

3. The medical device according to claim 2, further comprising a pressing tool capable of keeping the flexible portion pressed and releasing the press.

4. The medical device according to claim 3, wherein
the medical device further comprises:
a protrusion on the flexible portion;
guide grooves extending longitudinally on an outer surface; and
a pressing tool with a quasi-horseshoe-shaped section that has a right side plate and a left side plate extending downwards from both ends of a central plate toward the same side, and
part of the right side plate and part of the left side plate, respectively of the pressing tool engage the guide grooves, the pressing tool slides along a length of the medical device, and when the pressing tool is positioned at and around one end, an inner surface of the central plate of the pressing tool presses the protrusion to open a check valve, and when the pressing tool is positioned at and around the other end, the pressing tool does not press the protrusion.

5. The medical device according to claim 3, wherein
the medical device further comprises:
a protrusion on the flexible portion;
guide grooves extending circumferentially on an outer surface; and
an annular pressing tool having a protrusion key on part of an inner surface, and
the pressing tool engages the guide groove and is rotatable along the guide groove, and when the pressing tool is rotated to place the protrusion key on the protrusion of the flexible portion, the protrusion is pressed to open the check valve, and when the pressing tool is reversely rotated to separate the protrusion key from the protrusion of the flexible portion, the protrusion is not pressed.

6. The medical device according to claim 3, wherein
the pressing tool comprises a hollow cylinder and a cap,
the hollow cylinder is threaded and provided near the flexible portion,
the cap is threaded and threads into the hollow cylinder, and has a protrusion for pressing the flexible portion on a bottom surface of the cap,
when the cap is rotated, the cap is lowered to cause the protrusion to press the flexible portion to open the check valve, and
when the cap is rotated in a reverse direction, the cap does not press the flexible portion.

7. The medical device according to claim 3, wherein
the medical device further comprises a protrusion on the flexible portion,
the pressing tool comprises an elastic belt and engaging tools,
one end of the elastic belt is fastened near the protrusion, one of the engaging tools is mounted to the other end of the elastic belt, the other of the engaging tools is mounted to an outer surface of the medical device on a line connecting a fastening portion and the protrusion at one end of the elastic belt and on a side opposite the fastening portion seen from the protrusion, and
when the engaging tool at the other end of the elastic belt and the engaging tool at a frame are engaged, the elastic belt presses the protrusion to open the check valve.

8. The medical device according to any one of claims 1 to 7, wherein the valve body of the check valve is fitted to the outer peripheral surface of the small diameter pipe of the valve holder, and tightly contacts an inner surface of the medical device.

9. The medical device according to any one of claims 1 to 8, wherein the medical device is an outer needle (an indwelling needle) for a catheter, an inner needle for a catheter, a wing needle, a plug, or an infusion tube.

10. A medical side branch pipe in which a first straight pipe and a second straight pipe are arranged on a line, and one or more side branches intersect the first straight pipe and the second straight pipe at a confluence and communicate with each other, wherein at least one side branch has a check valve therein such that a fluid and a gas from a free end of the side branch to the confluence pass, but a fluid and a gas from the confluence to the free end are blocked and do not flow.

11. A medical side branch pipe in which a first straight pipe and a second straight pipe are arranged on a line, and one or more side branches intersect the first straight pipe and the second straight pipe at a confluence and communicate with each other, wherein at least one side branch has a check valve therein, part of a side surface of the side branch near the check valve is a flexible portion made of an elastic material, and when the flexible portion is not pressed, a fluid and a gas from a free end of the side branch to the confluence pass, but a fluid and a gas from the confluence to the free end are blocked and do not flow, while when the flexible portion is pressed, the fluid and the gas pass from the confluence to the free end.

12. The medical side branch pipe according to claim 11, further comprising a pressing tool capable of keeping the flexible portion pressed and releasing the press.

13. The medical side branch pipe according to any one of claims 10 to 12, wherein
the check valve comprises:
a valve holder that has a tapered pipe, and a small diameter pipe connecting to a small diameter opening of the tapered pipe and extending outwards, a large diameter opening of the tapered pipe being fluid-tightly fitted to an inner surface of a side branch; and
a flexible valve body that is fitted to an outer peripheral surface of the small diameter pipe of the valve holder, and has a slit that is opened by a fluid pressure from a large diameter opening side of the tapered pipe, or by a lateral pressure, but closed by a fluid pressure from a small diameter pipe side.

14. The medical side branch pipe according to any one of claims 10 to 13, further comprising a detachable side branch cap that is capable of closing a free end of at least one side branch.

15. The medical side branch pipe according to any one of claims 10 to 14, further comprising a detachable straight pipe cap that is capable of closing a free end of the first straight pipe.

16. The medical side branch pipe according to any one of claims 10 to 15, wherein the second straight pipe comprises a detachable straight pipe cap nut that is fluid-tightly connectable to the first straight pipe of the medical side branch pipe according to claim 15.

17. The medical side branch pipe according to any one of claims 10 to 16, wherein the second straight pipe comprises a detachable cap nut that is fluid-tightly connectable to the side branch of the medical side branch pipe according to any one of claims 10 to 16.

18. The medical side branch pipe according to any one of claims 2 to 7, 11 and 12, wherein the flexible portion is colored, or coated with fluorescent paint, or a color tape or a fluorescent paint tape is affixed to the flexible portion.

19. The medical device according to any one of claims 3 to 7, or claim 12, wherein a material for the pressing tool contains fluorescent paint, or the pressing tool is colored, or coated with the fluorescent paint, or a color tape, a fluorescent paint tape, or a reflection sheet is affixed to the pressing tool.
